SEQUENCE LISTING (43 sequences)

SEQ ID NO: 1 - protein, 10 aa, homo sapiens
MPSSVSWGIL

SEQ ID NO: 2 - protein, 10 aa, homo sapiens
LAGLCCLVPV

SEQ ID NO: 3 - protein, 10 aa, homo sapiens
SLAEDPQGDA

SEQ ID NO: 4 - protein, 10 aa, homo sapiens
AQKTDTSHHD

SEQ ID NO: 5 - protein, 10 aa, homo sapiens
QDHPTFNKIT

SEQ ID NO: 6 - protein, 10 aa, homo sapiens
PNLAEFAFSL

SEQ ID NO: 7 - protein, 10 aa, homo sapiens
YRQLAHQSNS

SEQ ID NO: 8 - protein, 10 aa, homo sapiens
TNIFFSPVSI

SEQ ID NO: 9 - protein, 10 aa, homo sapiens
ATAFAMLSLG

SEQ ID NO: 10 - protein, 10 aa, homo sapiens
TKADTHDEIL

SEQ ID NO: 11 - protein, 10 aa, homo sapiens
EGLNFNLTEI

SEQ ID NO: 12 - protein, 10 aa, homo sapiens
PEAQIHEGFQ

SEQ ID NO: 13 - protein, 10 aa, homo sapiens
ELLRTLNQPD

SEQ ID NO: 14 - protein, 10 aa, homo sapiens
SQLQLTTGNG

SEQ ID NO: 15 - protein, 10 aa, homo sapiens
LFLSEGLKLV

SEQ ID NO: 16 - protein, 10 aa, homo sapiens
DKFLEDVKKL

SEQ ID NO: 17 - protein, 10 aa, homo sapiens
YHSEAFTVNF

SEQ ID NO: 18 - protein, 10 aa, homo sapiens
GDHEEAKKQI

SEQ ID NO: 19 - protein, 10 aa, homo sapiens
NDYVEKGTQG

SEQ ID NO: 20 - protein, 10 aa, homo sapiens
KIVDLVKELD

SEQ ID NO: 21 - protein, 10 aa, homo sapiens
RDTVFALVNY

SEQ ID NO: 22 - protein, 10 aa, homo sapiens
IFFKGKWERP

SEQ ID NO: 23 - protein, 10 aa, homo sapiens
FEVKDTEDED

SEQ ID NO: 24 - protein, 10 aa, homo sapiens
FHVDQVTTVK

SEQ ID NO: 25 - protein, 10 aa, homo sapiens
VPMMKRLGMF

SEQ ID NO: 26 - protein, 10 aa, homo sapiens
NIQHCKKLSS

SEQ ID NO: 27 - protein, 10 aa, homo sapiens
WVLLMKYLGN

SEQ ID NO: 28 - protein, 10 aa, homo sapiens
ATAIFFLPDE

SEQ ID NO: 29 - protein, 10 aa, homo sapiens
GKLQHLENEL

SEQ ID NO: 30 - protein, 10 aa, homo sapiens
THDIITKFLE

SEQ ID NO: 31 - protein, 10 aa, homo sapiens
NEDRRSASLH

SEQ ID NO: 32 - protein, 10 aa, homo sapiens
LPKLSITGTY

SEQ ID NO: 33 - protein, 10 aa, homo sapiens
DLKSVLGQLG

SEQ ID NO: 34 - protein, 10 aa, homo sapiens
ITKVFSNGAD

SEQ ID NO: 35 - protein, 10 aa, homo sapiens
LSGVTEEAPL

SEQ ID NO: 36 - protein, 10 aa, homo sapiens
KLSKAVHKAV

SEQ ID NO: 37 - protein, 10 aa, homo sapiens
LTIDEKGTEA

SEQ ID NO: 38 - protein, 10 aa, homo sapiens
AGAMFLEAIP

SEQ ID NO: 39 - protein, 10 aa, homo sapiens
MSIPPEVKFN

SEQ ID NO: 40 - protein, 10 aa, homo sapiens
KPFVFLMIEQ

SEQ ID NO: 41 - protein, 10 aa, homo sapiens
NTKSPLFMGK

SEQ ID NO: 42 - protein, 7 aa, homo sapiens
VVNPTQK

SEQ ID NO: 43 - protein, 417 aa, homo sapiens
MPSSVSWGILLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

(19) United States
(12) Patent Application Publication
(10) Pub. No.: US 2008/0124303 A1
(43) Pub. Date: May 29, 2008

(54) Methods and compositions for treatment of viral infections

(75) Inventor: Colm King, Delray Beach, FL (US)

Correspondence Address: LAW OFFICES OF KHALILIAN SIRA, LLC, 9100 PERSIMMON TREE ROAD, POTOMAC, MD, 20854, US

(73) Assignee: Cavit Sciences, Inc

(21) Appl. No.: 11/827,491

(22) Filed: Jul. 12, 2007

Related U.S. Application Data
(63) Continuation-in-part of application No. 11/298,629, filed on Dec. 12, 2005.

(51) Int. Cl. A61K 38/21; A61K 31/715; A61K 31/704; A61K 36/00; A61K 36/06; A61P 31/04; A61P 31/12; A61K 39/395; A61K 35/74; A61K 38/00; A61K 38/16

(52) U.S. Cl. 424/85.6; 514/54; 514/33; 424/773; 424/195.15; 424/93.44; 424/725; 514/8; 424/93.4; 424/85.4; 424/158.1; 514/12

(57) ABSTRACT

A novel method of treating and preventing viral diseases is provided. In particular, the present invention relates to compositions and methods for inhibition of viral infections and the diseases associated with such viral infections. More particularly, the present invention relates to the inhibitory compounds comprising naturally occurring and man-made compositions comprising a substance exhibiting Tubercin and/or SSM activity or a functional derivative thereof.

### CROSS REFERENCE TO RELATED APPLICATIONS

This application is a continuation-in-part of U.S. application Ser. No. 11/298,629, filed Dec. 12, 2005, the entire contents of which are incorporated herein by reference.

### FIELD OF THE INVENTION

The present invention relates to compositions and methods for inhibition of viral infections and to therapeutic treatment of diseases or disorders caused by such viral infections. More particularly, the present invention also relates to inhibitory compounds comprising naturally occurring and man-made Tubercin, Specific Substance of Maruyama materials or a functional derivative thereof.

### BACKGROUND OF THE INVENTION

Human Immunodeficiency Virus

The human immunodeficiency virus (HIV) has been implicated as the primary cause of the slowly degenerative immune system disease termed acquired immune deficiency syndrome (AIDS) (Barre-Sinoussi, F., et al., 1983, Science 220:868-870; Gallo, R., et al., 1984, Science 224:500-503). There are at least two distinct types of HIV: HIV-1 (Barre-Sinoussi, F., et al., 1983, Science 220:868-870; Gallo, R., et al., 1984, Science 224:500-503) and HIV-2 (Clavel, F., et al., 1986, Science 233:343-346; Guyader, M., et al., 1987, Nature 326:662-669). Further, a large amount of genetic heterogeneity exists within populations of each of these types. In humans, HIV replication occurs prominently in CD4.sup.+T lymphocyte populations, and HIV infection leads to depletion of this cell type and eventually to immune incompetence, opportunistic infections, neurological dysfunctions, neoplastic growth, and ultimately death.

HIV is a member of the lentivirus family of retroviruses (Teich, N., et al., 1984, RNA Tumor Viruses, Weiss, R., et al., eds., CSH-Press, pp. 949-956). Retroviruses are small enveloped viruses that contain a single-stranded RNA genome and replicate via a DNA intermediate produced by a virally-encoded reverse transcriptase, an RNA-dependent DNA polymerase (Varmus, H., 1988, Science 240:1427-1439).

The HIV viral particle comprises a viral core, composed in part of capsid proteins, together with the viral RNA genome and those enzymes required for early replicative events. Myristylated gag protein forms an outer shell around the viral core, which is, in turn, surrounded by a lipid membrane envelope derived from the infected cell membrane. The HIV envelope surface glycoproteins are synthesized as a single 16.0 kilodalton precursor protein which is cleaved by a cellular protease during viral budding into two glycoproteins, gp41 and gp120. gp41 is a transmembrane glycoprotein and gp120 is an extracellular glycoprotein which remains non-covalently associated with gp41, possibly in a trimeric or multimeric form (Hammarskjold, M., & Rekosh, D., 1989, Biochem. Biophys. Acta 989:269-280).

HIV is targeted to CD4.sup.+ cells because a CD4 cell surface protein (CD4) acts as a cellular receptor for the HIV-1 virus (Dalgleish, A., et al., 1984, Nature 312:763-767; Klatzmann et al., 1984, Nature 312:767-768; Maddon et al., 1986, Cell 47:333-348). Viral entry into cells is dependent upon gp120 binding the cellular CD4 receptor molecules (McDougal, J. S., et al., 1986, Science 231:382-385; Maddon, P. J., et al., 1986, Cell 47:333-348), explaining HIV's tropism for CD4.sup.+ cells, while gp41 anchors the envelope glycoprotein complex in the viral membrane. While these virus:cell interactions are necessary for infection, there is evidence that additional virus:cell interactions are also required.

HIV Treatment

HIV infection is pandemic and HIV-associated diseases represent a major world health problem. Although considerable effort is being put into the design of effective therapeutics, currently no curative anti-retroviral drugs against AIDS exist. In attempts to develop such drugs, several stages of the HIV life cycle have been considered as targets for therapeutic intervention (Mitsuya, H., et al., 1991, FASEB J. 5:2369-2381). Many viral targets for intervention with HIV life cycle have been suggested, as the prevailing view is that interference with a host cell protein would have deleterious side effects. For example, virally encoded reverse transcriptase has been one focus of drug development. A number of reverse-transcriptase-targeted drugs, including 2′,3′-dideoxynucleside analogs such as AZT, ddI, ddC, tenofavir, neveripine, efavirenz, delavirdine, and d4T have been developed which have been shown to been active against HIV (Mitsuya, H., et al., 1991, Science 249:1533-1544).

The new treatment regimens for HIV-1 show that a combination of anti-HIV compounds, which target reverse transcriptase (RT), such as azidothymidine (AZT), lamivudine (3TC), dideoxyinosine (ddI), dideoxycytidine (ddC) used in combination with an HIV-1 protease inhibitor have a far greater effect (2 to 3 logs reduction) on viral load compared to AZT alone (about 1 log reduction). For example, impressive results have recently been obtained with a combination of AZT, ddI, 3TC and ritonavir (Perelson, A. S., et al., 1996, Science 15:1582-1586). However, it is likely that long-term use of combinations of these chemicals will lead to toxicity, especially to the bone marrow. Long-term cytotoxic therapy may also lead to suppression of CD8.sup.+T cells, which are essential to the control of HIV, via killer cell activity (Blazevic, V., et al., 1995, AIDS Res. Hum. Retroviruses 11:1335-1342) and by the release of suppressive factors, notably the chemokines Rantes, MIP-1.alpha. and MIP-1.beta. (Cocchi, F., et al., 1995, Science 270:1811-1815). Another major concern in long-term chemical anti-retroviral therapy is the development of HIV mutations with partial or complete resistance (Lange, J. M., 1995, AIDS Res. Hum. Retroviruses 10:S77-82). It is thought that such mutations may be an inevitable consequence of anti-viral therapy. The pattern of disappearance of wild-type virus and appearance of mutant virus due to treatment, combined with coincidental decline in CD4.sup.+T cell numbers strongly suggests that, at least with some compounds, the appearance of viral mutants is a major underlying factor in the failure of AIDS therapy.

Attempts are also being made to develop drugs which can inhibit viral entry into the cell, the earliest stage of HIV infection. Here, the focus has thus far been on CD4, the cell surface receptor for HIV. Recombinant soluble CD4, for example, has been shown to inhibit infection of CD4.sup.+T cells by some HIV-1 strains (Smith, D. H., et al., 1987, Science 238:1704-707). Certain primary HIV-1 isolates, however, are relatively less sensitive to inhibition by recombinant CD4 (Daar, E., et al., 1990, Proc. Natl. Acad. Sci. USA 87:6574-6579). In addition, recombinant soluble CD4 clinical trials have produced inconclusive results (Schooley, R., et al., 1990, Ann. Int. Med. 112:247-253; Kahn, J. O., et al., 1990, Ann. Int. Med. 112:254-261; Yarchoan, R., et al., 1989, Proc. Vth Int. Conf. on AIDS, p. 564, MCP 137).

The late stages of HIV replication, which involve crucial virus-specific processing of certain viral encoded proteins, have also been suggested as possible anti-HIV drug targets. Late stage processing is dependent on the activity of a viral protease, and drugs are being developed which inhibit this protease (Erickson, J., 1990, Science 249:527-533). Recently, chemokines produced by CD8.sup.+T cells have been implicated in suppression of HIV infection (Paul, W. E., 1994, Cell 82:177; Bolognesi, D. P., 1993, Semin. Immunol. 5:203). The chemokines RANTES, MIP-1.alpha. and MIP-1.beta., which are secreted by CD8.sup.+T cells, were shown to suppress HIV-1 p24 antigen production in cells infected with HIV-1 or HIV-2 isolates in vitro (Cocchi, F, et al., 1995, Science 270:1811-1815). Thus, these and other chemokines may prove useful in therapies for HIV infection. The clinical outcome, however, of all these and other candidate drugs is still in question.

Attention is also being given to the development of vaccines for the treatment of HIV infection. The HIV-1 envelope proteins (gp160, gp120, gp41) have been shown to be the major antigens for anti-HIV antibodies present in AIDS patients (Barin et al., 1985, Science 228:1094-1096). Thus far, therefore, these proteins appear to be the most promising candidates to act as antigens for anti-HIV vaccine development. Several groups have begun to use various portions of gp160, gp120, and/or gp41 as immunogenic targets for the host immune system. See for example, Ivanoff, L., et al., U.S. Pat. No. 5,141,867; Saith, G., et al., WO92/22,654; Shafferman, A., WO91/09,872, Formoso, C., et al., WO90/07,119. Vaccines directed against HIV proteins are problematic in that the virus mutates rapidly rendering many of these vaccines ineffective. Thus, although a great deal of effort is being directed to the design and testing of anti-retroviral drugs, effective, non-toxic treatments are still needed.

Because of some of the difficulties and inadequacies of conventional therapy for HIV-1, new therapeutic modalities are still desirable.

Herpesviridae

Herpes viruses are double stranded DNA viruses that replicate in host cell nuclei. The herpes virion is constituted from over 30 different proteins, which are assembled within the host cell. About 6-8 are used in the capsid. The preferred host cells for herpes viruses are vertebrate cells. The herpesviruses are animal viruses of significant clinical importance as they are the causative agents of many diseases. Epstein-Barr virus has been implicated in cancer initiation; cytomegalovirus (CMV) is the greatest infectious threat to AIDS patients; and Varicella Zoster Virus, is a causative agent of chicken pox and shingles. Herpes simplex virus subtypes 1 and 2 (HSV-1, HSV-2), are herpes viruses that are among the most common infectious agents encountered by humans. These viruses cause a broad spectrum of diseases, which range from relatively insignificant infections such as recurrent herpes simplex labialis, to severe and life-threatening diseases such as herpes simplex encephalitis. A large percentage of the United States population is affected by some form of a herpes virus infection. An estimated 98 million persons suffer each year from herpes labialis (HSV-1) and about 30 million cases of genital herpes (HSV-2) are recorded each year. Commonly these viruses are transmitted is by virus exposure at mucosal surfaces and abraded skin, permitting the entry of virus and viral replication in the epidermis and dermis. In addition to clinically apparent lesions, latent infections may persist, in particular in nerve cells. This is a difficult infection to eradicate. This scourge has largely gone unchecked due to the inadequacies of available treatment.

The vast majority of the human experience with these infections is associated with rather benign symptoms, such as malaise, fever, chills, rhinitis and diarrhea. However, herpes viruses are implicated in more serious health problems such as soft tissue sarcoma, carcinoma, metastatic disease, plasmacytoma, myeloma, lymphoma, certain heritable states including retinoblastoma, Li-Fraumeni syndrome, Gardner's syndrome, Werner's syndrome, nervoid basal cell carcinoma syndrome, neurofibromatosis type 1, and some immunodeficiency syndromes. Other conditions of notable clinical interest are leukoplakia, vesiculoulcerative mucosal diseases, idiopathic burning mouth, aphthous ulceration

For example, a single species of herpes family viruses, i.e., Epstein Barr virus (EBV) is associated with endemic Burkitt's lymphoma, acquired immune deficiency syndrome (AIDS)-related lymphoma, post-transplantation lymphoproliferative disease, Hodgkin's disease (HD), and rare T-cell lymphomas. Epstein-Barr virus is also associated with oral hairy leukoplakia, lymphoproliferative disease, lymphoepithelial carcinoma, B-cell lymphomas, and non-keratinising and squamous cell nasopharyngeal carcinoma.

Human herpesvirus-8 has been implicated in all forms of Kaposi's sarcoma, primary effusion lymphomas, multiple myeloma, angioimmunoblastic lymphadenopathy, and Castleman's disease. HHV-8 is also associated with certain lymphomas including rare B cell lymphomas called body-cavity-based lymphomas, epithelial tumors in kidney transplant recipients, malignant mesothelioma, angiosarcoma, and angiolymphoid hyperplasia.

Human herpesvirus-6 has been detected in and associated with lymphoproliferative disease, lymphomas, Hodgkin's disease, and oral squamous cell carcinoma.

Primary infection with HSV-1 rarely causes significant problems although widespread involvement in atopic eczema can be life-threatening as may associated encephalitis. Keratoconjunctivitis, pharyngitis and hepatitis can also complicate primary infection. Twenty to forty percent of the population at some stage have recurrent orolabial infections with HSV although in only one percent of these cases is this recurrence severe. Recurrent erythema multiforme appears to be associated with HSV-1 as sixty five percent of patients are thought to have preceding herpes labialis.

Herpes zoster infection may cause polyneuropathies, motor neuropathies, sensory neuronopathies, polyradiculoneuropathies, autonomic neuropathies, focal or multifocal cranial neuropathies, radiculopathies, and plexopathies, typically resulting from tumor infiltration.

People with acquired immunodeficiency syndrome (AIDS) are at an increased risk of Kaposi's sarcoma, non-Hodgkin's lymphoma, Hodgkin's disease, squamous cell carcinoma of the conjunctiva, and childhood leiomyosarcoma. It is striking that most of these cancers have been associated with specific human herpesvirus (HHV) infections: HHV-8 with Kaposi's sarcoma and the closely related Epstein-Barr virus with non-Hodgkin's lymphoma, Hodgkin's disease, and possibly also with childhood leiomyosarcoma. Moreover, similar associations between these viruses and cancer have been found, albeit inconsistently, in people who are not immunosuppressed. A general review on some aspects of herpesviridae-related diseases can be found in Flaitz and Hicks (Flaitz C M, Hicks M J. Molecular piracy: the viral link to carcinogenesis. Oral Oncol 1998 November; 34(6):448-53).

Because of some of the difficulties and inadequacies of conventional therapy for Herpes virus infections, new therapeutic modalities are still desirable.

Small Pox

Variola virus, the causative agent of smallpox, is a member of the Orthopoxvirus genus, which also includes monkeypox, cowpox, and vaccinia viruses. The disease caused by variola major strains is characterized by a low infectious dose (10-100 virions), long incubation period (averaging 12 days), fever, constitutional symptoms, rash progressing to a pustular stage, death in up to 30% of those affected, and facial scarring in survivors. The disease is spread person-to-person via the respiratory route by contact (droplets) and, possibly, by aerosol.

Smallpox was one of the most important causes of morbidity and mortality worldwide throughout the first half of the 20.sup.th century. However, in part because of the lack of animal reservoir for the virus, the systematic use of a vaccine (live, attenuated vaccinia virus) was highly effective in fighting this disease. Indeed, between 1967-1977, a global program of smallpox eradication resulted in the elimination of the natural disease (Fenner et al., WHO, Geneva, p. 1460, 1988). Because of the absence of smallpox and the risk of vaccine-associated adverse events, routine vaccination of children, hospital personnel, and military personnel has ceased, and only persons working with vaccinia and related viruses in the laboratory are currently immunized. Thus, a substantial portion of the world's population has no immunity to smallpox. The remaining population has little residual immunity, as vaccine immunity lasts only 5 years after primary vaccination and less than 20 years after revaccination. The eradication of smallpox and the cessation of vaccination have, thus, created vulnerability in the population to covert attack or biowarfare employing variola virus. Should such an event occur, epidemic spread would be unchecked by an immune barrier in the population (Anon. (Editorial), Lancet 353:1539, 1999; Henderson, Science 283:1279-1282, 1999; Henderson et al., J.A.M.A. 281:2127-2137, 1999).

Because of the uncertainties surrounding smallpox eradication, vaccine was stockpiled for emergency use. In the United States, for example, 155,000 vaccine vials (nominally 15.5 million doses) produced by Wyeth Laboratories were originally stockpiled under the control of the Centers for Disease Control and Prevention (CDC), Atlanta, Ga., U.S. At a meeting of the National Vaccines Advisory Committee in January 1999, the CDC reported on the status of the national smallpox vaccine repository. At that time, of the 15.5 million doses held by Wyeth, 3.4 million doses had failed quality control testing and 10.3 million were beyond the expiration date specified by the last control test for extended dating, leaving 1.7 million doses that met release specifications (LeDuc, Presentation to the National Vaccines Advisory Committee, Washington D.C., Jan. 11-12, 1999). In addition to the limited supply, the vaccine is packaged in 100 dose vials, which restricts distribution and increases the likelihood of wastage during an emergency.

In addition to the U.S. stockpile, there is a supply of vaccine (Lister, Elstree strain) stored at the National Institute of Public Health, Bilthoven, Netherlands, and certain other countries have supplies of smallpox vaccine, which at the time of eradication may have included up to 300 million doses. However, similar problems of stability in storage have reduced this supply to less than 50 million doses (Henderson, Science 283:1279-1282, 1999).

Accordingly, despite some successful therapies using a variety of anti-viral agents and/or nucleoside analogues additional and improved treatments directed against these other viral targets are still desperately needed.

This invention addresses a long-felt need for safe and effective compositions and methods of treatment of viral infections.

### SUMMARY OF THE INVENTION

The present invention relates to therapeutically active compounds, pharmaceutical formulations containing said compounds and the use of said compounds in treatment and prophylaxis, particularly of viral infections.

In a preferred embodiment, the therapeutically active compound that inhibits viral infection or production in a mammal comprises Tubercin, Tubercin-3, Tubercin-5, Tubercin-7, SSM (otherwise known as SSMA or Specific Substance of Maruyama), or Z-100 or any combination thereof. In yet another preferred embodiment, the agent that inhibits viral infection or production in a mammal comprises a Tubercin, Tubercin-3, Tubercin-5, Tubercin-7, SSM, or Z-100-based oligosaccharide-protein conjugate or lipid arabinomannan-protein conjugate or any combination thereof.

The agent that inhibits viral infection or production in a mammal can include, but is not limited to, small organic molecules including naturally-occurring, synthetic, and biosynthetic molecules, small inorganic molecules including naturally-occurring and/or synthetic molecules.

One aspect of the present invention is to provide clinically acceptable viral inhibitors exhibiting relatively high anti viral activity at relatively low concentrations.

The invention further provides pharmaceutical compositions comprising such agents.

The present invention provides methods for treating viral infections in a mammal comprising administering to a subject in need thereof a therapeutically effective amount of a composition comprising a substance exhibiting Tubercin and/or SSM activity or a functional derivative thereof; and a pharmaceutically acceptible excipient.

Also provided is a method of inhibiting a viral infection of a mammal, which comprises administering to a mammal susceptible to a viral infection an effective amount of a substance exhibiting Tubercin and/or SSM activity or a functional derivative thereof; and a pharmaceutically acceptible excipient.

In one embodiment, the virus inhibited from infecting a mammal comprises a DNA virus from the genus comprising hepadnaviridae, adenoviridae, parvoviridae, papovariridae, poxyiridae, iridoviridae, and herpesviridae or any combination thereof.

In another embodiment, the virus inhibited from infecting a mammal comprises an RNA virus from the genus comprising picornaviridae, calciviridae, togaviridae, flaviviridae, coronaviridae, rhabdoviridae, filoviridae, paramyxoviridae, orthomyxoviridae, bunyaviridae, arenaviridae, reoviridae, and bimaviridae or any combination thereof.

In yet another embodiment, the virus inhibited from infecting a mammal comprises a virus from the genus comprising lentiviridae including HTLV-I, HTLV-II, HTLV-III (HIV-1) and HIV-2 and hepatitis viruses including hepatitis A, B, C, delta, E and/or G virus, or any combination thereof.

In one embodiment, the methods of the present invention are used to prevent or ameliorate a symptom of AIDS. In one embodiment, the methods of the present invention are used to prevent or ameliorate a symptom of AIDS selected from the group consisting of malaise, fever, dry cough, myalgias, and chest pains, ventilatory compromise, sweating, fever, abdominal pain, diarrhea, and mucosal ulcerations or any combination thereof.

In another embodiment, the specific viral diseases inhibited in a mammal by administering an effective amount of a substance exhibiting Tubercin and/or SSM activity or a functional derivative thereof comprise, inter alia, viral hepatitis (A, B, C, delta, E and G), influenza, viral pneumonia, viral bronchitis, herpetic infections (herpes simplex virus, Epstein Barr virus (infectious mononucleosis), herpes zoster (varicella zoster Virus (VZV)), poliomyelitis, AIDS (HIV-1 infection), adult T-cell leukemia (ATL), papilloma (HPV), measles, rubella, exanthema subitum, erythema infectiosum, viral encephalitis, viral myelitis, Visna (Sheep) and Equestrian Anemia, cytomegalovirus infection, mumps, varicella, rabies, viral enteritis, viral myocarditis, viral pericarditis or any combination thereof.

In another embodiment, the viruses inhibited from infecting a mammal by administering an effective amount of a substance exhibiting Tubercin and/or SSM activity or a functional derivative thereof comprise SARS-associated coronavirus (SARS-CoV), avian influenza (bird flu), and avian influenza A (H5N1), or any combination thereof.

In another embodiment, the virus inhibited from infecting a mammal comprises a virus from the genus comprising Filoviridae including, Ebola-Zaire, Ebola-Sudan, and Ebola-Ivory Coast, and Ebola-Reston, or any combination thereof.

Also provided is a method of inhibiting an opportunistic or bacterial infection in a mammal, which comprises administering to a mammal susceptible to an opportunistic or bacterial infection an effective amount of a substance exhibiting Tubercin and/or SSM activity or a functional derivative thereof; and a pharmaceutically acceptible excipient.

In another embodiment, the bacterial infection inhibited from infecting a mammal comprises Methicillin-resistant Staphylococcus Aureus (MRSA) or any variants thereof.

In another embodiment, the bacterial opportunistic infection inhibited from infecting a mammal comprises Candidiasis (Thrush), Chronic mucocutaneous candidiasis, Candida vulvovaginitis, Dry mouth (xerostomia), Diabetes mellitus, Cytomegalovirus (CMV), Herpes simplex viruses, Oral herpes, Genital herpes, Herpes Zoster, Malaria, Mycobacterium avium complex (MAC or MAI), Pneumocystis pneumonia (PCP), Toxoplasmosis (Toxo), Tuberculosis (TB), Pneumocystis jirovecii, Pneumocystis carinii, Candida albicans infection, Staphylococcus aureus, Streptococcus pyogenes, Streptococcus pneumoniae, Pseudomonas aeruginosa, Acinetobacter baumanni, Toxoplasma gondii, Cytomegalovirus, Cytomegalovirus retinitis, Aspergillus sp., Aspergillosis, Penicillium marneffei, Miliary TB, Invasive candidiasis, Histoplasmosis, Disseminated strongyloidiasis, Legionellosis, Respiratory syscytial virus, Hepatitis, Hepatitis A, Hepatitis B, Salmonellosis, Syphilis and Neuroshyphilis, Bacillary angiomatosis, Coccidioidomycosis, Cryptococcal meningitis, Kaposi's Sarcoma, Systemic Non-Hodgkin's Lymphoma, Primary CNS lymphoma, Cryptosporidiosis, Isosporiasis, Microsporidiosis, Human Papiloma Virus (HPV, genital warts, cervical cancer), Molluscum Contagiosum, Oral Hairy Leukoplakia (OHL), Progressive Multifocal Leukoencephalopathy (PML), AIDS Dementia Complex (ADC), Peripheral Neuropathy, Apthous Ulcers, Malabsorption, or any variants thereof.

In another embodiment, the opportunistic infection inhibited from infecting a mammal comprises Systemic inflammatory response syndrome (SIRS), E. faecalis, Methicillin-resistant Staphylococcus aureus (MRSA), CLP-induced sepsis, Salmonella typhimurium, Trypanosoma cruzi, Lymphocytic choriomeningitis virus, Listeria monocytogenes, Herpetic encephalomyelitis, Cryptococcal encephalitis, Human cytomegalovirus, Herpes simplex virus type 2, Influenza virus, Coronavirus, HIV, Poliovirus, Newcastle disease virus, Vesicular stomatitis virus, Ehrlich carcinoma, Sarcoma-180, or any variants thereof.

In another embodiment, the specific symptoms and/or diseases inhibited in a mammal by administering an effective amount of a substance exhibiting Tubercin and/or SSM activity or a functional derivative thereof comprise, inter alia, malaise, thrush, night sweats and flu like systems, fever, chills, rhinitis, diarrhea, atopic eczema, encephalitis, keratoconjunctivitis, pharyngitis, gingivostomatitis, herpetic hepatitis, recurrent orofacial mucocutaneous lesions or herpes labialis, shingles, small pox skin sores, chicken pox skin sores, erythema multiforme, idiopathic burning mouth, aphthous ulceration, Behcet's syndrome, mononucleosis, Burkitt's lymphoma, primary effusion lymphomas, multiple myeloma, angioimmunoblastic lymphadenopathy, Castleman's disease, acquired immune deficiency syndrome (AIDS)-related lymphoma, post-transplantation lymphoproliferative disease, Hodgkin's disease, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, malignant mesothelioma, angiosarcoma, Kaposi's sarcoma, angiolymphoid hyperplasia, prostatic neoplasm, cervical cancer, neoplasms of the vulva, retinoblastoma, Li-Fraumeni syndrome, Gardner's syndrome, Werner's syndrome, nervoid basal cell carcinoma syndrome, neurofibromatosis type 1, polyneuropathies, motor neuropathies, sensory neuronopathies, polyradiculoneuropathies, autonomic neuropathies, focal or multifocal cranial neuropathies, radiculopathies, plexopathies typically resulting from tumor infiltration, chronic urinary tract infections, vaginosis, vaginitis, cervical dysplasia, genital warts, plantar warts, sexually or perinatally transmitted herpes disease, or any combination thereof.

In another embodiment, the Tubercin and/or SSM activity or a functional derivative thereof may be used, inter alia, in immonocompromised hosts as an inducer of suppressor cell antagonists or a regulator of IL-4 produced by suppressor cells, to inhibit the growth of viruses, to potentiate certain events associated with immunities, to induce interferon, to promote tumor regression, to prolong life span, to extend survival rate, as an antitumor effect, to modulate immune responses in hosts, for its ability to counter immunodeficient situations, in modulating the host's biological response to tumor cells, to induce interferon in patients with cancer, in combination with biological response modifiers (BRM's), as a potential immunotherapeutic anticancer agent, in activation of the reticuloendothelial system, for protection against X-ray-induced leucopenia, to augment natural killer (NK) cell activity, and/or to generate cytostatic macrophages.

In another embodiment, the specific symptoms and/or diseases inhibited in a mammal by administering an effective amount of a substance exhibiting Tubercin and/or SSM activity or a functional derivative thereof comprise Severe acute respiratory syndrome (SARS), Ebola hemorrhagic fever (Ebola HF), and Methicillin-resistant Staphylococcus Aureus (MRSA), or any combination thereof.

In yet another aspect of the present invention, the specific diseases and/or conditions either inhibited in a mammal or capable of being treated in a mammal by administering an effective amount of a substance exhibiting Tubercin and/or SSM activity or a functional derivative thereof comprise transmissable agents including the progressive, degenerative neurological diseases known as transmissible spongiform encephalopathies (TSEs). In certain embodiments, the transmissible spongiform encephalopathies (TSEs) comprise Scrapie, Chronic wasting disease (CWD), Bovine Spongiform Encephalopathy (BSE) (sometimes referred to as “mad cow disease”) and variant and classic Creutzfeldt-Jakob disease (CJD) or any combination thereof.

In bne embodiment, the reduction or inhibition of pain and/or symptoms associated with one or more of each of the above-recited viral diseases and/or indications is on the order of about 10-20% reduction or inhibition. In another embodiment, the reduction or inhibition of pain is on the order of 30-40%. In another embodiment, the reduction or inhibition of pain is on the order of 50-60%. In yet another embodiment, the reduction or inhibition of the pain associated with each of the recited viral diseases and/or indications is on the order of 75-100%. It is intended herein that the ranges recited also include all those specific percentage amounts between the recited range. For example, the range of about 75 to 100% also encompasses 76 to 99%, 77 to 98%, etc, without actually reciting each specific range therewith.

It is thus another aspect of the present invention to provide a novel method for treating a retroviral infection which comprises administering to a host in need thereof a therapeutically effective combination of (a) a substance exhibiting Tubercin and/or SSM activity or a functional derivative thereof and (b) one or more compounds selected from the group consisting of retroviral reverse transcriptase inhibitors, retroviral protease inhibitors, or an entry inhibitor. Accordingly, reverse transcriptase inhibitor can be selected from a group including nucleoside RT inhibitors: Retrovir (AZT/zidovudine; Glaxo Wellcome); Combivir (Glaxo Wellcome); Epivir (3TC, lamivudine; Glaxo Wellcome); Videx (ddI/didanosine; Bristol-Myers Squibb); Hivid (ddC/zalcitabine; Hoffmann-La Roche); Zerit (d4T/stavudine; Bristol-Myers Squibb); Ziagen (abacavir, 1592U89; Glaxo Wellcome); tenofovir, emtricitabine, Hydrea (Hydroxyurea/HO; nucleoside RT potentiator from Bristol-Myers Squibb) or Non-nucleoside reverse transcriptase inhibitors (NNRTIs): Viramune (nevirapine; Roxane Laboratories); Rescriptor (delavirdine; Pharmacia & Upjohn); Sustiva (efavirenz, DMP-266; DuPont Merck); Preveon (adefovir dipivoxil, bis-POM PMEA; Gilead). Protease inhibitors (PI's) are selected from Fortovase (saquinavir; Hoffmann-La Roche); Norvir (ritonavir; Abbott Laboratories); Crixivan (indinavir; Merck & Company); Viracept (nelfinavir; Agouron Pharmaceuticals); Angenerase (amprenavir/141W94; Glaxo Wellcome), atazanavir, Kaletra (lopinavir/ritonavir) VX-478, KNI-272, CGP-61755, and U-103017, or the entry inhibitor T20 (Fuzeon or enfuvirtide), or any combination thereof.

For each of the above-recited methods of the present invention, the therapeutically effective amount of the one or more substances exhibiting Tubercin and/or SSM activity or a functional derivative thereof and a pharmaceutically acceptible excipient thereof may be administered to a subject in need thereof in conjunction with a therapeutically effective amount of one or more anti-viral drugs and/or inflammatory compounds and/or a therapeutically effective amount of one or more immunomodulatory agents.

In certain embodiments of the methods of the present invention, the anti-inflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof, soluble TNF-receptors; TNF-antibodies; soluble receptors of interleukins, other cytokines, T-cell-proteins; antibodies against receptors of interleukins, other cytokines, T-cell-proteins; and calcipotriols and analogues thereof taken either alone or in any combination thereof.

In yet another aspect, the present invention is directed to a method of relieving or ameliorating the pain or symptoms associated with any one or more of the above-identified viral diseases and/or indications in a mammal suffering from any one or more of the above-identified viral diseases or indications which comprises administering to the mammal in need thereof a therapeutically effective pain or symptom-reducing amount of a pharmaceutical composition comprising effective amounts of a substance exhibiting Tubercin and/or SSM activity or a functional derivative thereof and a pharmaceutically acceptible excipient, either alone or in combination with one or more anti-inflammatory compounds or immunomodulatory agents, wherein said substance exhibiting Tubercin and/or SSM activity or a functional derivative thereof is sufficient to inhibit the viral disease and/or indication.

The present invention also relates to the combined use of the pharmaceutical composition comprising a substance exhibiting Tubercin and/or SSM activity or a functional derivative thereof and a pharmaceutically acceptible excipient in combination with one or more antibacterial or antiviral compositions or any combination thereof for treating any one of the aforementioned viral diseases and/or indications, or any combination thereof.

For each of the above-recited methods of the present invention, the therapeutically effective amount of the one or more substances exhibiting Tubercin and/or SSM activity and/or Z-100 activity or a functional derivative thereof and a pharmaceutically acceptable excipient thereof may be administered to a subject in need thereof in conjunction with a therapeutically effective amount of one or more immune enhancers or a functional derivative thereof.

In yet another embodiment of the present invention, a method is provided for inhibiting an opportunistic, a bacterial infection or any one or more of the viral infections, viral diseases or viral indications listed supra in a mammal, which comprises administering to a mammal susceptible to an opportunistic, a bacterial infection or any one or more of the viral infections, viral diseases or viral indications a therapeutically effective amount of one or more immune enhancers or a functional derivative thereof; and a pharmaceutically acceptible excipient, wherein said one or more immune enhancers or a functional derivative thereof is sufficient to inhibit the opportunistic, the bacterial infection, or any one or more of the viral infections, viral diseases or viral indications in a mammal.

In yet another embodiment of the present invention, a method is provided for modulating (e.g., stimulate or inhibit) the immune system in a mammal which comprises administering to a mammal a therapeutically effective amount of one or more immune enhancers or a functional derivative thereof; and a pharmaceutically acceptable excipient, wherein said one or more immune enhancers or a functional derivative thereof is sufficient to modulate the immune system in a mammal.

In certain embodiments of the methods of the present invention, the immune enhancers comprise thymosin and interleukins, contact dermatitis inducers, cyclosporin and inosiplex, bacterial lysates of S. aureus, BCG mycobacteria, Corynebacterium Baruum and levamizole, interleulin-1, or other interleukin molecules, an interferon, or muramil dipeptide or tripeptide, unmethylated CpG dinucleotides, CD40 ligand, complement C3d fragment, B cell activating factor, soluble gene product of lymphocyte activation gene-3, Early Secretory Antigenic Target 6 (ESAT-6) polypeptide, and analogues thereof taken either alone or in any combination thereof.

In certain embodiments of the methods of the present invention, the immune enhancers comprise Glycyrrhizin (GR), consisting of one molecule of glycyrrhetic acid and two molecules of glucuronic acid, is an active component of Glycyrrhiza glabra roots (Immunology Letters, 44 (i995) 59-66; Current Drug Targets, 2006, 7, 119-134, Bacterial Sepsis and Chemokines and Antimicrobial Agents and Chemotherapy, mar. 1997, p. 551-556), Ge-132, Carboxyethylgermanium sesquioxide, a synthesized organogermanium compound (Microbial. Immununol. Vol. 29 (1), 65-74, 1985, Birkhauser Verlag basel, 1996; In Vivo 1: 189-204 (1987), SMANCS. (polystyrene-co-maleic acid conjucated neocarzinostatin)(Anticancer Research 8: 97-104 (1988)), Krestin (PSK), hot water extract of mycelium of Criolus versicolor (U.S. Pat. No. 5,254,534), Picibanil (OK-432), a whole cell preparation of streptococcus pyogenes (Cancer Immunol. Immunother. 6, 41-45 (1979)), OK-432 article (U.S. Pat. No. 5,254,534), Lentinan, a B-1,3-glucan extracted from fruit body of Lentinus ebodes (U.S. Pat. No. 5,254,534), KS-2 (peptidomannan), Journal of Antibiotics, November 1978; Journal of Antibiotics, December 1979), Muramyl dipeptide (active component of LPS) (In vivo Gen product sheet and J Biol Chem 2003), ATSO, an antitumor polysaccharide from Coriolus versicolor (Journal of the Pharmaceutical Society of Japan, Vol. 97 and No. 2 (1977)), Levamisole (synthesized compound)(Cellular Immunology 43, 214-219 (1979)), 9-Methylstreptimidone (antibiotics)(The New York Academy of Sciences, Vol 284, Pages 667-675, Mar. 4, 1977), Schizophyllan, a polysaccharide derived from culture medium of Schizophyllum commune (U.S. Pat. No. 5,254,534), Corynebacterium parvum (Letters to Nature 257, 396-398 (2 Oct. 1975); Infect Immun. 1981 June; 32(3): 1128-1131), Mushroom extract, aqueous extract of Lentinus edodes, a kind of Japanese edible mushroom (Mushroom Science, 1974), Dextran phosphate (Proceedings of the Society for Experimental Biology and Medicine 149, 1069-1075 (1975)), TH69, a lyophilized whole cell preparation of Streptococcus faecalis (Gann, 73, 811-818; October, 1982), Shigyaku-to (TJS-109), traditional Chinese herbal medicine (Birkhauser verlag Basel, 1994), Kanzo-bushi-to (TJS-038) one of the Japanese Kampo medicines originating from traditional Chinese herbal medicine (Immunology Letters, 40 (1994) 13-20), Keishi-Ka-Kei-To, a traditional Chinese herbal medicine (Anticancer Research 17:873-878 (1997)), and analogues or functional derivatives thereof either alone or any combination thereof (the disclosures of each of these publications and patents in their entireties are hereby incorporated by reference).

The present invention thus provides methods for therapeutically or prophylactically treating viral infections and/or viral indications in a subject.

In one embodiment, the method for therapeutically treating viral infections comprises the step of administering pharmaceutically effective amounts of a substance exhibiting Tubercin and/or SSM activity or a functional derivative thereof and a pharmaceutically acceptible excipient to the subject after occurrence of the viral disease and/or viral indication.

In another embodiment, the method for prophylactically treating viral infections comprises the step of administering pharmaceutically effective amounts of a substance exhibiting Tubercin and/or SSM activity or a functional derivative thereof and a pharmaceutically acceptible excipient to the subject prior to the occurrence of the viral disease and/or viral indication.

Either methodology inhibits the viral infection of a mammal.

In one embodiment, with respect to the use of the compositions and methods of the present invention to prevent, ameliorate or treat a symptom caused by either HIV-1, hepatitis virus, or murine herpes virus, specifically excluded within the scope of the present invention is the use of Z-100 as disclosed in the reference of Yutaka et al. (Inhibition of human immunodeficiency virus type 1 replication by Z-100, an immunomodulator extracted from human-type tubercle bacilli, in macrophages Yutaka Emoril et al. J Gen Virol 85 (2004), 2603-2613)).

In yet another embodiment, with respect to the use of the compositions and methods of the present invention to prevent, ameliorate or treat a symptom of herpes virus type 1, specifically excluded within the scope of the present invention is the use of Z-100 as disclosed in the reference of Kobayashi M et al. (Lipid-arabinomannan extracted from Mycobacterium tuberculosis, improves the resistance of thermally injured mice to herpes virus infections (Kobayashi M et al. Immunol Lett. 1994 June; 40 (3):199-205)).

The preferred doses for administration can be anywhere in a range between about 10 ng and about 10 mg per ml or mg of the formulation. The therapeutically effective amount of a substance exhibiting Tubercin and/or SSM activity or a functional derivative thereof can be also measured in molar concentrations and may range between about 1 nM and about 10 mM. The formulation is also contemplated in combination with a pharmaceutically or cosmetically acceptable carrier. The precise doses can be established by well known routine clinical trials without undue experimentation.

In yet another aspect, the present invention provides a method for preventing a symptom of a given viral infection in a subject thought to be at risk for exposure to a given viral infection comprising admininstering to the subject a pharmaceutically effective amount of a substance exhibiting Tubercin and/or SSM activity or a functional derivative thereof, wherein said substance exhibiting Tubercin and/or SSM activity or a functional derivative thereof substance inhibits the attachment of a given virus to one or more viral receptors, and wherein if the subject is exposed to the virus, a symptom of said exposure is prevented.

In another aspect, the present invention provides a method for preventing a symptom of a given viral infection in a subject suspected of having been exposed to a given viral infection comprising admininstering to the subject a pharmaceutically effective amount of a substance exhibiting Tubercin and/or SSM activity or a functional derivative thereof, wherein said substance exhibiting Tubercin and/or SSM activity or a functional derivative thereof inhibits the attachment of a given virus to one or more viral receptors, and wherein if the subject is exposed to the virus, a symptom of said exposure is prevented.

In another aspect, the present invention provides a method for ameliorating a symptom of a given viral infection in a subject in need of said amelioration comprising admininstering to the subject a pharmaceutically effective amount of a substance exhibiting Tubercin and/or SSM activity or a functional derivative thereof, wherein said substance exhibiting Tubercin and/or SSM activity or a functional derivative thereof inhibits the attachment of a given virus to one or more viral receptors.

Also a method is contemplated that reduces the likelihood of herpes infection in a susceptible individual in occupational and non-occupational settings by providing post-exposure prophylaxis. A similar aim of reducing viral infection is accomplished by providing an effective antiviral dose of a Tubercin and/or SSM compound or a functional derivative thereof into an oral, rectal and/or vaginal cavity to prevent sexual transmission of herpes and/or preventing or inhibiting in utero transmission.

As a derivation of this preferred embodiment a method of reducing or preventing herpes virus replication in a patient is provided which consists of administering a therapeutically effective amount of Tubercin and/or SSM compound or a functional derivative thereof in combination with other compounds, e.g., nucleoside drugs like acyclovir, that display anti-herpes virus activity.

The invention also encompasses methods for the treatment of pre-existing lesions and sores of the skin or mucosa associated with a herpes virus infection and for prevention of future lesions and sores of the skin or mucosa associated with a herpes virus infection, which comprise administering any one or more of the above-described compositions in a pharmaceutically effective amount for the treatment and/or prevention of these lesions.

Also provided herein is a general method of treating a mammal suffering from a pathological condition that is mediated by viral infection is contemplated as well, which comprises administering a therapeutically effective amount of a substance exhibiting Tubercin and/or SSM activity or a functional derivative thereof. This pathological condition, e.g., inflammatory reaction, tumorigenesis, autoimmune disease, etc., can result directly or indirectly from said viral infections.

A novel medical treatment and medicine is provided to quickly and safely resolve herpes and other viral infections. The topical formulation can be applied and maintained on the infected region until the physical symptoms of the disease disappear and the patient is comfortable and has a normal appearance.

It should be apparent that in addition to these aforementioned preferred embodiments a method is contemplated which consists of treating an individual having superficial viral infection or a physiological condition caused, in whole or part, by a superficial virus infection of skin, mucosal surface which lines the body cavities. Examples of mucosal surface which can be infected with herpes viruses include infections of the oral soft tissues; middle ear; gastrointestinal tract; urogenital tract; airway/lung tissue, eye; and peritoneal membranes.

In accordance with this embodiment a method of inhibiting topically viral infection or treating topically a condition is provided wherein the targets of the therapy are tissues and organs indicated supra which organs are contacted with an effective amount of a compound having Tubercin and/or SSM activity or a functional derivative thereof for a sufficient amount of time.

Among preferred compounds to treat such viruses are substantially purified natural or synthetic Tubercin and/or SSM compounds or a functional derivatives thereof. Tubercin and/or SSM and similarly active compounds may be identified by a series of assays wherein a compound (natural or synthetic Tubercin and/or SSM or a functional derivative thereof) will exhibit viral inhibitory activity versus control in an assay. For example, and not by way of limitation, one of these assays comprises blocking interleukin-18 or IL-18-induced human immunodeficiency virus (HIV) production in U1 monocytic cells. Other assays involve blocking stimulants such as IL-6, NaCl, LPS, TNF, and other HIV stimuli known in the art. Other assays involve MAGI-CCR-5 cell assay and the PBMC assay as described in detail in the body of this disclosure. Other similar viral inhibitory-based assays known to those of skill in the art may be used to identify natural or synthetic Tubercin and/or SSM compounds or a functional derivatives thereof for use in any one of the aforementioned methods of the present invention.

The treatment and prevention of virus-induced tumors by administering the substance exhibiting Tubercin and/or SSM activity or a functional derivative thereof is yet another aspect of this invention. Yet another preferred embodiment of this invention is to provide a substance exhibiting Tubercin and/or SSM activity or functional derivative thereof for treatment of various types of cancer that may or may not be virus-induced but are capable of metastasizing. Such tumors may comprise fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, rhabdosarcoma, colorectal carcinoma, pancreatic cancer, breast cancer, ovarian cancer, prostate cancer, melanoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, myeloma, lymphoma, and leukemia or any combination thereof.

In yet another aspect of the present invention a method is provided for treating and/or preventing viral infections in a mammal comprising admisinistering Tubercin and/or SSM in combination with one more proteins or peptides. In the aspect of this invention pertaining to the Tubercin and/or SSM-protein/peptide combination therapy, among the preferred compounds to treat any one of the aforementioned viruses, viral indications and/or diseases is a substantially purified natural or recombinant protein or peptide. Such proteins or peptide fragments thereof can be associated with one or more modified saccharides or modified oligosaccharides using chemical synthesis methods known to those of skill in the art so as to form a glycoprotein or glycopolypeptide. Representative examples of classes of proteins which can be used as the non-saccharide portion of a molecule include antibodies, enzymes, growth factors, cytokines and chemokines. Antibodies which can be associated with a modified saccharide, as described herein, include CDP-571, gemtuzumab ozogamicin, biciromab, imciromab, capromab, .sup.111 indium satumomab pendetide, bevacizumab, ibritumomab tiuxetan, cetuximab, sulesomab, afelimomab, HuMax-CD4, MDX-RA, palivizumab, basiliximab, inolimomab, lerdelimumab, pemtumomab, idiotypic vaccine (CEA), Titan, Leucotropin, etanercept, pexelizumab, alemtuzumab, natalizumab, efalizumab, trastuzumab, epratuzumab, palivizumab, daclizumab, lintuzumab, Cytogam, Engerix-B, Enbrel, Gamimune (IgG), Meningitec, Rituxan, Synagis, Reopro, Herceptin, Sandoglobulin, Menjugate, and BMS-188667. Growth factors, enzymes and receptors which can be used as non-saccharide moieties include Benefix, Meningitec, Refacto, Procit, Epogen, Eprex, Intron A, Neupogen, Humulin, Avonex, Betaseron, Cerezyme, Genotropin, Kogenate, NeoRecormon, Gonal-F, Humalog, NovoSeven, Puregon, Norditropin, Rebif, Nutropin, Activase, Espo, Neupogen, Integrilin, Roferon, Insuman, Serostim, Prolastin, Pulmozyme, Granocyte, Creon, Hetrodin HP, Dasen, Saizen, Leukine, Infergen, Retavase, Proleukin, Regranex, Z-100, somatropin, Humatrope, Nutropin Depot, somatropin, epoetin delta, Eutropin, ranpimase, infliximab, tifacogin, oprelvekin, interferon-alpha, aldesleukin, OP-1, drotrecogin alfa, tasonermin, oprelvekin, etanercept, afelimomab, daclizumab, thymosin alpha 1, becaplermin, and A-74187. Other non-saccharide moieties which can be used include pexelizumab, anakinra, darbepoetin alfa, insulin glargine, Avonex, alemtuzumab, Leucotropin, Betaseron, aldesleukin, domase alfa, tenecteplase, oprelvekin, choriogonadotropin alfa, and nasaruplase, or any combination thereof.

In yet another aspect, the present invention entails a novel method of treating and/or preventing viral infections facilitated by a serine proteolytic (SP) activity comprising administering to a subject suffering or about to suffer from said viral infection a therapeutically effective amount of a compound having a serine protease inhibitory or serpin activity or a compound exhibiting mammalian α1-antitrypsin (AAT) or AAT-like activity comprising α1-antitrypsin activity (AAT) in combination with a pharmaceutically effective amount of a substance exhibiting Tubercin and/or SSM activity or a functional derivative thereof, wherein said combination therapy effectively inhibits viral replication, attachment of the virus to one or more viral receptors and/or one or more viral symptoms and/or indications.

In the aspect of this invention pertaining to the Tubercin and/or SSM-AAT combination therapy, the substance exhibiting Tubercin and/or SSM activity or functional derivative thereof may be either mixed together in one formulation or the Tubercin and/or SSM activity compound or functional derivative thereof may be administered in a therapeutically effective amount either before, contemporaneously or subsequent to administration of the therapeutically effective amount of a compound having a serine protease inhibitory or serpin activity or a compound exhibiting mammalian α1-antitrypsin (AAT) or AAT-like activity comprising α1-antitrypsin activity (AAT).

In the aspect of this invention pertaining to the Tubercin and/or SSM-AAT combination therapy, the substance exhibiting Tubercin and/or SSM activity or a functional derivative thereof may also be directly conjugated to the AAT compound or functional derivative thereof using either routine or advanced synthetic chemical reactions known to those of skill in the chemical art.

In the aspect of this invention pertaining to the Tubercin and/or SSM-AAT combination therapy, among the preferred compounds to treat any one of the aforementioned viruses, viral indications and/or diseases is a substantially purified natural or recombinant AAT. Preferably, AAT is substantially purified from a wild type, mutant, or transgenic mammalian source or isolated from a culture of wild type, mutant, or transformed cells.

The peptides of interest are homologous and analogous peptides. While homologues are natural peptides with sequence homology, analogues will be peptidyl derivatives, e.g., aldehyde or ketone derivatives of such peptides. Without limiting to AAT and peptide derivatives of AAT, the compounds like oxadiazole, thiadiazole and triazole peptoids and substances comprising certain phenylenedialkanoate esters are preferred.

In each of the above-recited methods, the mammalian α1-antitrypsin or inhibitor of serine protease activity substance contemplated for use in the Tubercin and/or SSM-AAT combination therapy methods of the present invention further comprises a series of peptides comprising amino acid peptides corresponding to portions or fragments of AAT. For example, and not by way of limitation, amino acid peptides corresponding to 10 amino acid fragments of AAT are specifically contemplated for use in the compositions and methods of the present invention. In particular, amino acid peptides MPSSVSWGIL (SEQUENCE ID NO. 19); LAGLCCLVPV (SEQUENCE ID NO. 20) SLAEDPQGDA (SEQUENCE ID NO. 21); AQKTDTSHHD (SEQUENCE ID NO. 22) QDHPTFNKIT (SEQUENCE ID NO. 23); PNLAEFAFSL (SEQUENCE ID NO. 24); YRQLAHQSNS (SEQUENCE ID NO. 25); TNIFFSPVSI (SEQUENCE ID NO. 26); ATAFAMLSLG (SEQUENCE ID NO. 27); TKADTHDEIL (SEQUENCE ID NO. 28); EGLNFNLTEI (SEQUENCE ID NO. 29); PEAQIHEGFQ (SEQUENCE ID NO. 30); ELLRTLNQPD (SEQUENCE ID NO. 31); SQLQLTTGNG (SEQUENCE ID NO. 32); LFLSEGLKLV (SEQUENCE ID NO. 33); DKFLEDVKKL (SEQUENCE ID NO. 34); YHSEAFTVNF (SEQUENCE ID NO. 35); GDHEEAKKQI (SEQUENCE ID NO. 36); NDYVEKGTQG (SEQUENCE ID NO. 37); KIVDLVKELD (SEQUENCE ID NO. 38); RDTVFALVNY (SEQUENCE ID NO. 39); IFFKGKWERP (SEQUENCE ID NO. 40); FEVKDTEDED (SEQUENCE ID NO. 41); FHVDQVTTVK (SEQUENCE ID NO. 42); VPMMKRLGMF (SEQUENCE ID NO. 43); NIQHCKKLSS (SEQUENCE ID NO. 44); WVLLMKYLGN (SEQUENCE ID NO. 45); ATAIFFLPDE (SEQUENCE ID NO. 46); GKLQHLENEL (SEQUENCE ID NO. 47); THDIITKFLE (SEQUENCE ID NO. 48); NEDRRSASLH (SEQUENCE ID NO. 49); LPKLSITGTY (SEQUENCE ID NO. 50); DLKSVLGQLG (SEQUENCE ID NO. 51); ITKVFSNGAD (SEQUENCE ID NO. 52); LSGVTEEAPL (SEQUENCE ID NO. 53); KLSKAVHKAV (SEQUENCE ID NO. 54); LTIDEKGTEA (SEQUENCE ID NO. 55); AGAMFLEAIP (SEQUENCE ID NO. 56); MSIPPEVKFN (SEQUENCE ID NO. 57); KPFVFLMIEQ (SEQUENCE ID NO. 58); NTKSPLFMGK (SEQUENCE ID NO. 59); VVNPTQK (SEQUENCE ID NO. 60), or any combination thereof.

Moreover, it is specifically intended that the AAT peptides contemplated for use in the Tubercin and/or SSM-AAT combination therapy methods of the present invention are also intended to include any and all of those specific AAT peptides other than the 10 amino acid AAT peptides of SEQ ID NO. 1 depicted supra. For example, while AAT peptides amino acids 1-10, amino acids 11-20, amino acids 21-30, etc of SEQ ID NO. 1 have been enumerated herein, it is specifically intended herein that the scope of the compositions and methods of use of same specifically include all of the possible combinations of AAT peptides such as amino acids 2-12, amino acid 3-13, 4-14, etc. of SEQ ID NO. 1, as well as any and all AAT peptide fragments corresponding to select amino acids of SEQ ID NO. 1, without actually reciting each specific AAT peptide of SEQ ID NO. 1 therewith. Thus, by way of illustration, and not by way of limitation, Applicants are herein entitled to possession of compositions based upon any and all AAT peptide variants based upon the amino acid sequence depicted in SEQ ID NO. 1 and use of such compositions in the methods of the present invention.

In each of the above-recited methods, the substance exhibiting Tubercin and/or SSM activity or a functional derivative thereof may be part of a fusion polypeptide, wherein said fusion polypeptide comprises a substance exhibiting Tubercin and/or SSM activity or a functional derivative thereof and an amino acid sequence heterologous to said substance exhibiting Tubercin and/or SSM activity or a functional derivative thereof.

In certain embodiments, the fusion polypeptide contemplated for use in the methods of the present invention comprises a human immunoglobin constant region, such as for example, a human IgG1 constant region, including a modified human IgG1 constant region wherein the IgG1 constant region does not bind the Fc receptor and/or does not initiate antibody-dependent cellular cytotoxicity (ADCC) reactions.

In yet other embodiments, the fusion polypeptides contemplated for use in the methods of the present invention can additionally comprise an amino acid sequence that is useful for identifying, tracking or purifying the fusion polypeptide, e.g., the fusion polypeptide can further comprise a FLAG or HIS tag sequence. The fusion polypeptide can additionally further comprise a proteolytic cleavage site which can be used to remove the heterologous amino acid sequence from substance exhibiting Tubercin and/or SSM activity or a functional derivative thereof.

In yet another aspect of the present invention, for each of the above-recited methods, the one or more substances exhibiting Tubercin and/or SSM activity or a functional derivative thereof may themselves be administered as an adjuvant, wherein the therapeutically effective amount of the one or more substances exhibiting Tubercin and/or SSM activity or a functional derivative thereof acts as an immunostimulant or immunomodulator to be used in conjunction with one or more of the pharmaceutical drugs listed in the Physicians Desk Reference as described infra.

In yet another aspect of the present invention, for each of the above-recited methods of the present invention, the therapeutically effective amount of the one or more substances exhibiting Tubercin and/or SSM activity or a functional derivative thereof may themselves be administered as an adjuvant in vaccine preparations to improve vaccine responses to all known bacterial, viral or parasitic antigen preparations, wherein the therapeutically effective amount of the one or more substances exhibiting Tubercin and/or SSM activity or a functional derivative thereof acts as an immunostimulant or immunomodulator to be used in conjunction with one or more of the pharmaceutical drugs listed in the Physicians Desk Reference as described infra.

In each of the above-recited methods, the substance exhibiting Tubercin and/or SSM activity or a functional derivative that inhibits a viral infection in a mammal can comprise small organic molecules or compounds including naturally-occurring, synthetic, and biosynthetic molecules or compounds, small inorganic molecules or compounds including naturally-occurring and/or synthetic molecules or compounds provided that said molecules or compounds specifically exhibit Tubercin and/or SSM activity or Tubercin and/or SSM-like activity as can be tested by the in vitro assays described in detail infra.

In yet another aspect of the present invention, for each of the above-recited methods or indications the present invention also includes homeopathic and/or holistic preparations of Tubercin, SSM and/or Z-100 or a functional derivative thereof to treat any one or more of the aforementioned viruses, viral indications, opportunistic infections and/or diseases. The homeopathic and/or holistic preparations of the present invention typically comprise between 1×10⁻⁶ and 1×10⁻⁵ molar concentrations of Tubercin, SSM and/or Z-100 or a functional derivative thereof in a pharmaceutically acceptable diluent. Various diluents may be used, depending on the appropriate delivery system. Appropriate diluents for the following delivery systems are well known: oral administration in liquid or solid form, intravenous administration; injection; eye drops; nasal sprays; and topical administration. One or more potencies of a specified growth factor and/or one or more growth factors may be combined in a preparation. The preferred homeopathic and/or holistic diluent for oral administration is a solution of purified water, glycerin, citric acid and a preservative such as sodium benzoate. Other diluents for oral delivery, including various alcohol-containing solutions, are known in the art and may be employed in the present invention to increase solubility and stability of growth factors. The homeopathic and/or holistic dilutions of the present invention are preferably administered orally, but may also be prepared in topical formulations for application to the skin, administered transdermally, administered in the form of eye drops, injected into acupuncture or skin conductance points, or administered intravenously. In a preferred embodiment, homeopathic and/or holistic dilutions of Tubercin, SSM and/or Z-100 or a functional derivative thereof are administered by means of liquids or tablets which retain the memory of the homeopathic and/or holistic dilution. The tablets are made from a suitable organic material, such as lactose (Botanical Labs., Bellingham, Wash.) by methods well known in homeopathy and/or holistic medicine (see, for example, the United States Homeopathic Pharmacopeia, the contents of which are specifically incorporated herein by reference in their entirety).

In one aspect of the invention, the pharmaceutical compositions of the present invention are administered orally, systemically, via an implant, intravenously, topically, intrathecally, intracranially, intraventricularly, by inhalation or nasally.

In certain embodiments of the methods of the present invention, the subject or mammal is a human.

In other embodiments of the methods of the present invention, the subject or mammal is a veterinary and/or a domesticated mammal.

While the invention has been illustrated using Tubercin, SSM and functional derivateives thereof for each of the above-mentioned methods, it should be readily apparent that each of the aforementioned methods may be practiced without undue experimentation using other bacterial cell wall extracts that have been previously used as immune stimulants and anti-tumor agents. Accordingly, representative examples of such bacterial cell wall extracts that may be used in each of the aforementioned methods are all those bacterial species from the genera Mycobacterium, Propionibacterium, Nocardia, and Actinomycetes, as well as Bacillus Calmette-Guerin (BCG), polysaccharide K, Beta 1,3-glucan, and extracts of Bifidobacterium, L. Lactis, L. fermentum, L. acidopholus, and S. lactis, as if specifically set forth herein in their entirety. For example, and not by way of limitation, muramyl peptidyl glycan complex (MPGC) is a non-toxic bacterial cell wall extract of Lactobacillus fermentum that contains muramic acid moieties attached to variable length mannose-rich polysaccharides. The mannose rich polysaccharides promote internalization of the entire muramic acid-containing complex and may be used in each of the methods of the present invention.

There has been thus outlined, rather broadly, the important features of the invention in order that a detailed description thereof that follows can be better understood, and in order that the present contribution can be better appreciated. There are additional features of the invention that will be described hereinafter.

In this respect, before explaining at least one embodiment of the invention in detail, it is to be understood that the invention is not limited in its application to the details as set forth in the following description and figures. The present invention is capable of other embodiments and of being practiced and carried out in various ways. Additionally, it is to be understood that the terminology and phraseology employed herein are for the purpose of description and should not be regarded as limiting.

As such, those skilled in the art will appreciate that the conception, upon which this disclosure is based, can readily be used as a basis for designing other methods for carrying out the several features and advantages of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates the effect of Tubercin in U1 cells.

FIG. 2 further illustrates the effect of Tubercin and lipopolysaccharide in U1 cells.

FIG. 3 shows proliferation and toxicity studies conducted in U1 cells.

FIG. 4 illustrates the effect of Tubercin in human peripheral blood mononuclear cells (PBMC) infected with HIV.

FIG. 5 shows the results of quantification of IL-8 in one of the PBMC cultures shown in FIG. 4.

FIG. 6 shows the results of IL-6 measured in the same cultures as described for FIG. 5.

FIG. 7 shows the results of Tubercin inhibition of HIV-1 infection in MAGI cells.

### DETAILED DESCRIPTION OF THE INVENTION

Standard Methods

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Therapeutic Methods

The present invention provides methods for treating viral infections comprising administering to a subject in need thereof of a therapeutically effective amount of a composition comprising an effective amount of a substance exhibiting Tubercin and/or SSM activity or a functional derivative thereof; and a pharmaceutically acceptible excipient.

Therefore, administration of a dosage of the invention composition, i.e., substance exhibiting Tubercin and/or SSM activity or a functional derivative thereof, can be beneficial for the treatment of viral diseases or disorders. In a preferred aspect, the agent is an analog of a substance exhibiting Tubercin and/or SSM activity or a functional derivative thereof that can cross the blood brain barrier, which would allow for intravenous or oral administration. Many strategies are available for crossing the blood brain barrier, including but not limited to, increasing the hydrophobic nature of a molecule; introducing the molecule as a conjugate to a carrier, such as transferrin, targeted to a receptor in the blood brain barrier; and the like. In another embodiment, the agent can be administered intracranially or, more directly, intraventricularly. In yet another embodiment, the agent can be administered by way of inhalation or nasally.

In a further embodiment, the methods and compositions of the invention are useful in the therapeutic treatment of viral diseases or disorders of the immune system. In a yet further embodiment, diseases can be prevented by the timely administration of the agent of the invention as a prophylactic, prior to onset of symptoms, or signs, or prior to onset of severe symptoms or signs of a viral disease. Thus, a patient at risk for a particular viral disease can be treated with a substance exhibiting Tubercin and/or SSM activity or a functional derivative thereof as a precautionary measure.

The effective dose of the agent of the invention, and the appropriate treatment regime, can vary with the indication and patient condition, and the nature of the molecule itself, e.g., its in vivo half life and level of activity. These parameters are readily addressed by one of ordinary skill in the art and can be determined by routine experimentation.

The preferred doses for administration can be anywhere in a range between about 1 picogram/ml and about 500 ug/ml of biologic fluid of treated patient. The therapeutically effective amount of the substance exhibiting Tubercin and/or SSM activity or a functional derivative thereof that has similar antiviral activities as substance exhibiting Tubercin and/or SSM activity or a functional derivative thereof can be also measured in molar concentrations and can range between about 1 nM to about 2 mM.

Viral Diseases Addressed by the Invention

Specific viral diseases or disorders for which the therapeutic methods of inhibiting the viral infection in a mammal of the invention are beneficial include, but are not limited to, those viral diseases or disorders caused by DNA viruses, RNA viruses and retroviruses.

In one embodiment, the virus inhibited from infecting a mammal comprises a DNA virus from the genus comprising hepadnaviridae, adenoviridae, parvoviridae, papovariridae, poxyiridae, iridoviridae, and herpesviridae.

In another embodiment, the virus inhibited from infecting a mammal comprises an RNA virus from the genus comprising picornaviridae, calciviridae, togaviridae, flaviviridae, coronaviridae, rhabdoviridae, filoviridae, paramyxoviridae, orthomyxoviridae, bunyaviridae, arenaviridae, reoviridae, and bimaviridae.

In yet another embodiment, the virus inhibited from infecting a mammal comprises a virus from the genus comprising lentiviridae including HTLV-I, HTLV-II, HTLV-III (HIV-1) and HIV-2 and hepatitis viruses including hepatitis A, B, C, delta and/or E virus or any combination thereof.

Viral Diseases and/or Symptoms Treatable by the Methods of the Present Invention

In another embodiment, the specific viral diseases inhibited in a mammal by administering an effective amount of a substance exhibiting Tubercin and/or SSM activity or a functional derivative thereof comprise, inter alia, viral hepatitis (A, B, C, E), influenza, viral pneumonia, viral bronchitis, herpetic infections (herpes simplex virus, Epstein Barr virus (infectious mononucleosis), herpes zoster), poliomyelitis, AIDS (HIV-1 infection), adult T-cell leukemia (ATL), papilloma, measles, rubella, exanthema subitum, erythema infectiosum, viral encephalitis, viral myelitis, cytomegalovirus infection, mumps, varicella, rabies, viral enteritis, viral myocarditis, viral pericarditis and so on.

In one embodiment, the methods of the present invention are used to prevent or ameliorate a symptom of AIDS. In one embodiment, the methods of the present invention are used to prevent or ameliorate a symptom of AIDS selected from the group consisting of malaise, fever, dry cough, myalgias, and chest pains, ventilatory compromise, sweating, widening of the mediastimum on radiographic studies, edema of the neck and chest, necrotizing mediastinal lymphadenitis, non-pitting edema, eschar, nausea, vomiting, fever, abdominal pain, bloody diarrhea, mucosal ulcerations, and hemorrhagic mesenteric lymphadenitis, or any combination thereof.

In another embodiment, the specific symptoms and/or diseases inhibited in a mammal by administering an effective amount of a substance exhibiting Tubercin and/or SSM activity or a functional derivative thereof comprise, inter alia, malaise, fever, chills, rhinitis, diarrhea, atopic eczema, encephalitis, keratoconjunctivitis, pharyngitis, gingivostomatitis, herpetic hepatitis, recurrent orofacial mucocutaneous lesions or herpes labialis, small pox skin sores, chicken pox skin sores, erythema multiforme, idiopathic burning mouth, aphthous ulceration, Behcet's syndrome, mononucleosis, Burkitt's lymphoma, primary effusion lymphomas, multiple myeloma, angioimmunoblastic lymphadenopathy, Castleman's disease, acquired immune deficiency syndrome (AIDS)-related lymphoma, post-transplantation lymphoproliferative disease, Hodgkin's disease, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, malignant mesothelioma, angiosarcoma, Kaposi's sarcoma, angiolymphoid hyperplasia, prostatic neoplasm, cervical cancer, neoplasms of the vulva, retinoblastoma, Li-Fraumeni syndrome, Gardner's syndrome, Werner's syndrome, nervoid basal cell carcinoma syndrome, neurofibromatosis type 1, polyneuropathies, motor neuropathies, sensory neuronopathies, polyradiculoneuropathies, autonomic neuropathies, focal or multifocal cranial neuropathies, radiculopathies, plexopathies typically resulting from tumor infiltration, sexually or perinatally transmitted herpes disease, or combinations thereof.

Thus, in view of the above, the present invention provides methods for preventing a symptom of AIDS in a subject suspected of having been exposed to or thought to be at risk for exposure to HIV-1 comprising admininstering to the subject a pharmaceutically effective amount of a substance exhibiting Tubercin and/or SSM activity or a functional derivative thereof. The present invention also provides a method for ameliorating a symptom of AIDS in a subject in need of said amelioration comprising admininstering to the subject a pharmaceutically effective amount of a substance exhibiting Tubercin and/or SSM activity or a functional derivative thereof.

Compositons for Use in the Methods of the Present Invention

Tubercin

Tubercin-3 and Tubercin-5

The Tubercin compositions that may be used in the methods of the present inventioin comprises those tubercin carbohydrate complexes designated Tubercin-3 and Tubercin-5. Tubercin-3 may be prepared as described in Chung, T. H., J. Korean Med. As., 17, 427-431 (1974); Chung, T. H. et al., Yonsei Med. J., 17, 131-135 (1976). Tubercin-5 may be extracted from M. tuberculosis as described in U.S. Pat. No. 6,274,356, the full contents of which are incorporated herein by reference. Tubercin-5 is a mixture of polysaccharides having straight-chain and side-chain glycosidic bonds formed between such essential monosaccharides as mannose, arabinose, glucose and galactose. The molecular weight of said polysacchrides lies below 7,000, preferably in the range of 2,500 to 3,500 dalton.

For example, the Tubercin-5 carbohydrate complex consists of an extract of Mycobacterium tuberculosis consisting essentially of polysaccharides comprising mannose, arabinose, glucose and galactose as constituents thereof, wherein the weight average molecular weight of each polysaccharide is 7,000 or less and the partial acid hydrolysis product of the polysacharides comprises:

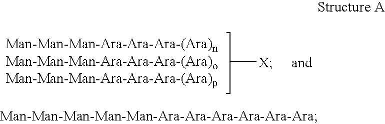

wherein n, o and p are individually an integer; and x is a chain of glucose and galactose residues.

In another embodiment, the partial acid hydrolysis product of the extract of Tubercin-5 depicted in Structure A can further comprise

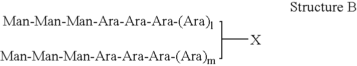

wherein 1 and m are individually an integer; and x is a chain of glucose and galactose residues.

In yet another embodiment, the partial acid hydrolysis product of the extract of Tubercin-5 depicted in Structure B can comprise

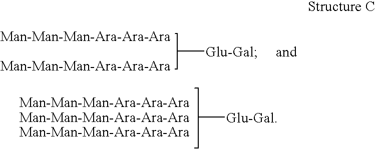

In yet another embodiment, the partial acid hydrolysis product of the extract of Tubercin-5 depicted in Structure A can further comprise partial acid hydrolysis products of any of the above, including, but not limited to:
Man-Man;
Man-Man-Man;
Man-Man-Man-Ara;
Man-Man-Man-Ara-Ara;
Man-Man-Man-Man-Ara-Ara-Ara-Ara;
Man-Man-Man-Ara-Ara-Ara-Ara-Ara-Ara; and
Man-Man-Man-Man-Ara-Ara-Ara-Ara-Ara-Ara.

Specific Substance of Maruyama (SSM)

SSM refers to a vaccine derived from a strain of tuberculosis, which gets its name, Specific Substance of Maruyama, from the late Professor Chisato Maruyama of Tokyo's Nippon Medical School. SSM (sometimes referred to herein as SSMA) comprises arabinomannan as a polysaccharide and has fatty acids bonded to said arabinomannan through an ester linkage, the fatty acid content in said lipopolysaccharide being from 3 to 28%. The lipopolysaccharide is obtained by hot water extraction and purification of the cell body of human tubercle bacillus, Mycobacterium tuberculosis strain Aoyama B or Mycobacterium tuberculosis strain H.sub.37 R.sub.v SSM is thus a lipoarabinomannan and comprises a lipopolysaccharide structure having definite chemical composition, which may be isolated and purified as described in either U.S. Pat. No. 4,394,502, the full contents of which are incorporated herein by reference, or U.S. Pat. No. 4,329,452, the full contents of which are incorporated herein by reference. In some embodiments, the SSM lipopolysaccharide may be prepared by bonding a fatty acid to arabinomannan through an ester linkage, said arabinomannan being obtained by alkali extraction and purification of the cell body of human tubercle bacillus, Mycobacterium tuberculosis strain Aoyama B or Mycobacterium tuberculosis strain H.sub.37 R.sub.v, the fatty acid content in said lipopolysaccharide being 3 to 28%. In other embodiments, the SSM lipopolysaccharide may be prepared by bonding a fatty acid to lipoarabinomannan through an ester linkage, said lipoarabinomannan being obtained by hot water extraction and purification of the cell body of human tubercle bacillus, Mycobacterium tuberculosis strain Aoyama B or Mycobacterium tuberculosis strain H.sub.37 R.sub.v, the fatty acid content in said lipopolysaccharide being 3 to 28%. In either case, the fatty acids are palmitic acid, myristic acid, stearic acid, tuberculostearic acid, heptadecanoic acid, oleic acid and linoleic acid and said lipopolysaccharide has a monosaccharide composition of 30 to 74% of arabinose, 20 to 50% of mannose, 0 to 10% of glucose and 0 to 13% of galactose.

In a study to examine the effect of SSM on clearance of HBe antigen, Satomura K et al. demonstrated that SSM stimulates the production of IFN-gamma in human peripheral blood cells and also suggest that treatment of HBe antigen-positive chronic hepatitis B patients with SSM leads to the clearance of HBe antigen and normalization of serum aspartate aminotransferase levels through inhibition of IL-10 and stimulation of IFN-gamma. (Effects of SSM (specific substance maruyama) on HBe antigen-positive chronic hepatitis B-clinical efficacy and modulation of cytokines J Nippon Med Sch. 2000 August; 67(4):261-6).

Z-100

Z-100 is a more powerful version of SSM. Z-100 is the same agent as SSM, but used in different concentrations. Z-100 is an arabinomannan extracted from Mycobacterium tuberculosis that has various immunomodulatory activities, such as the induction of interleukin 12, interferon gamma (IFN-) and -chemokines (Inhibition of human immunodeficiency virus type 1 replication by Z-100, an immunomodulator extracted from human-type tubercle bacilli, in macrophages Yutaka Emoril et al. J Gen Virol 85 (2004), 2603-2613). Yutaka et al. investigated the effects of Z-100 on human immunodeficiency virus type 1 (HIV-1) replication in human monocyte-derived macrophages (MDMs). In MDMs, Z-100 markedly suppressed the replication of not only macrophage-tropic (M-tropic) HIV-1 strain (HIV-1JR-CSF), but also HIV-1 pseudotypes that possessed amphotropic Moloney murine leukemia virus or vesicular stomatitis virus G envelopes. Yutaka et al. also demonstrated that Z-100 was found to inhibit HIV-1 expression, even when added 24 h after infection. In addition, Yutaka et al. also demonstrated that Z-100 substantially inhibited the expression of the pNL43lucenv vector (in which the env gene is defective and the nef gene is replaced with the firefly luciferase gene) when this vector was transfected directly into MDMs. Together, these findings suggest that Z-100 inhibits virus replication, mainly at the level of HIV-1 transcription. However, Yutaka et al. also demonstrated that Z-100 also downregulated expression of the cell surface receptors CD4 and CCR5 in MDMs, suggesting some inhibitory effect on HIV-1 entry. Further experiments by Yutaka et al. demonstrated that Z-100 revealed that Z-100 induced IFN-production in these cells, resulting in induction of the 16-kDa CCAAT/enhancer binding protein (C/EBP) transcription factor that represses HIV-1 long terminal repeat transcription. These effects were alleviated by SB 203580, a specific inhibitor of p38 mitogen-activated protein kinases (MAPK), indicating that the p38 MAPK signalling pathway was involved in Z-100-induced repression of HIV-1 replication in MDMs. Taken together, these findings of Yutaka et al. suggest that Z-100 might be a useful immunomodulator for control of HIV-1 infection.

Moreover, the effect of Z-100, a lipid-arabinomannan extracted from Mycobacterium tuberculosis strain Aoyama B, was investigated on the resistance of thermally injured mice (TI-mice) to herpes simplex virus type 1 (HSV) infections Z-100 (Kobayashi M et al. Lipid-arabinomannan extracted from Mycobacterium tuberculosis, improves the resistance of thermally injured mice to herpes virus infections (Kobayashi M et al. Immunol Lett. 1994 June; 40(3): 199-205). Kobayashi M et al. demonstrated that the susceptibility of TI mice to infection was about 100 times greater than it was in normal mice (N mice). However, the increased susceptibility of TI mice to infection was effectively counteracted to the levels observed in N mice when treated with Z-100 (10 mg/kg i.p.; 1, 3 and 5 days after thermal injury). Kobayashi M et al. demonstrated that adoptive transfer of burn-associated CD8+ CD11b+TCR gamma/delta+suppressor T (BAST) cells, prepared from TI mice, increased the susceptibility of N mice to infection by HSV, while the susceptibility of N mice, inoculated with the CD8+ T-cell fraction prepared from Z-100-treated TI mice (ZTC), to infection was not changed. In addition, Kobayashi M et al. demonstrated that the suppressor cell activity of BAST cells was not demonstrated when they were assayed in vitro in the presence of anti-IL-4 monoclonal antibody (mAb). BAST cells released IL-4 into their culture fluids without stimulation. The suppressor cell activity of ZTC and IL-4 production by ZTC were minimal. Taken together, the results of Kobayashi M et al. demonstrated that Z-100 may improve the resistance of TI mice to HSV infection through the regulation of BAST cells and/or the release of IL-4 from these cells.

In addition to the Tubercin and SSM (Z-100) compounds and/or substances disclosed supra, in yet another embodiment, the SSM and/or Tubercin composition for use in all of the aforementioned methods of the present invention may be a SSM and/or Tubercin functional derivative composition comprising, inter alia, a polysaccharide produced by a hot aqueous solvent extraction of tubercle bacillus, wherein the polysaccharide is comprised of arabinose, mannose and glucose residues. The SSM and/or Tubercin functional derivative composition for use in the methods of the present invention may be further described in that the polysaccharide has a molecular weight of about 5.times.10.sup.2-5.times.10.sup.4, as determined by gel filtration. In one embodiment, the polysaccharide of the SSM/Tubercin functional derivative composition of the present invention is comprised of 10-72 wt. % mannose, 3-30 wt. % of arabinose and 5-30% wt. % of glucose. In another embodiment, the polysaccharide of the SSM/Tubercin functional derivative composition of the present invention is comprised of 40-50 wt. % mannose, 15-25 wt. % of arabinose and 5-15% wt. % of glucose. The SSM/Tubercin functional derivative composition may be prepared and isolated as more fully described in U.S. Pat. No. 6,015,796. It is intended herein that the ranges recited also include all those specific percentage amounts between the recited range. For example, the range of about 10 to 72% also encompasses 11 to 71%, 12 to 708%, etc, without actually reciting each specific range therewith.

Serine Protease Inhibitors For Use in the Tubercin and/or SSM-AAT Combination Therapy

Another aspect of the present invention entails a novel method of treating and/or preventing viral infections facilitated by a serine proteolytic (SP) activity comprising administering to a subject suffering or about to suffer from said viral infection a therapeutically effective amount of a compound having a serine protease inhibitory or serpin activity or a compound exhibiting mammalian alpha-1-antitrypsin (AAT) or AAT-like activity comprising α1-antitrypsin activity (AAT) in combination with a pharmaceutically effective amount of a substance exhibiting Tubercin and/or SSM activity or a functional derivative thereof, wherein said combination therapy effectively inhibits viral replication, attachment of the virus to one or more viral receptors and/or one more more viral symptoms and/or indications.

By using the substance exhibiting Tubercin and/or SSM activity or a functional derivative thereof in combination with mammalian alpha-1-antitrypsin (AAT) or AAT-like activity comprising α1-antitrypsin activity (AAT) to ameliorate and/or treat viral infections in a mammal suffering from one or more viral infections, several advantages are obtained compared to alternative approaches, for example, and not by way of limitation:

1. Synthetic inhibitors of serine proteases (AAT-like mimics) can and have been developed (See, infra, CE-2072). Such a pharmaceutical agent may be formulated into a pill for oral consumption in the field or formulated as an inhaler to treat those viral diseases spreadable by means of inhalation.

2. Commercially available agents already approved for alternate use in humans will work as a treatment for viral infections. These agents are currently used for indications other than viral infections, and include injectible AAT, plasma preparations, aprotinin and others (American J. Of Resp Critical Care Med 1998, VII 158: 49-59). One possible instantiation of this invention may be of immediate practical application. For example, and not way of limitation, inhibitors of serine proteases have been delivered to patients by inhalation.

3. This approach to anti-viral therapy is likely to be safe. There is an extensive clinical experience using injectible AAT to treat patients with genetic AAT deficiency. No long-term untoward effects have been detected to date (American J. Of Resp Critical Care Med 1998, VII 158: 49-59; Wencker et al. Chest 2001 119:737-744). Moreover, a small molecule inhibitor of host serine protease has been administered to patients with Kawasaki's Disease (Ulinistatin, Ono pharmaceuticals), with an excellent safety and tolerability record. In addition, inhibition of host serine proteases to treat viral infection will only require a short treatment course, thus minimizing any potential concerns with long term exposure to AAT or AAT-like mimics/or other inhibitors of serine protease.

Accordingly, for those embodiments of the present invention that utilize the alpha-1-antitrypsin (AAT) or AAT-like activity comprising α1-antitrypsin activity (AAT) in combination with a pharmaceutically effective amount of a substance exhibiting Tubercin and/or SSM activity or a functional derivative thereof, the serine proteases and inhibitors of serine protease that are contemplated for use are outlined in detail below.

Serine Proteases and Serine Protease Inhibitors

Serine proteases serve an important role in human physiology by mediating the activation of vital functions. In addition to their normal physiological function, serine proteases have been implicated in a number of pathological conditions in humans. Serine proteases are characterized by a catalytic triad consisting of aspartic acid, histidine and serine at the active site.

The naturally occurring serine protease inhibitors are usually, but not always, polypeptides and proteins which have been classified into families primarily on the basis of the disulfide bonding pattern and the sequence homology of the reactive site. Serine protease inhibitors, including the group known as serpins, have been found in microbes, in the tissues and fluids of plants, animals, insects and other organisms. Protease inhibitor activities were first discovered in human plasma by Fermi and Pemossi in 1894. At least nine separate, well-characterized proteins are now identified, which share the ability to inhibit the activity of various proteases. Several of the inhibitors have been grouped together, namely α1-antitrypsin-proteinase inhibitor, antithrombin III, antichymotrypsin, C1-inhibitor, and α2-antiplasmin, which are directed against various serine proteases, i.e., leukocyte elastase, thrombin, cathepsin G, chymotrypsin, plasminogen activators, and plasmin. These inhibitors are members of the α1-antitrypsin-proteinase inhibitor class. The protein α2-macroglobulin inhibits members of all four catalytic classes: serine, cysteine, aspartic, and metalloproteases. However, other types of protease inhibitors are class specific. For example, the α1-antitrypsin-proteinase inhibitor (also known as (α1-antitrypsin or AAT) and inter-alpha-trypsin inhibitor inhibit only serine proteases, α1-cysteine protease inhibitor inhibits cysteine proteases, and a 1-anticollagenase inhibits collagenolytic enzymes of the metalloenzyme class.

Human neutrophil elastase (NE) is a proteolytic enzyme secreted by polymorphonuclear leukocytes in response to a variety of inflammatory stimuli. The degradative capacity of NE, under normal circumstances, is modulated by relatively high plasma concentrations of α1-antitrypsin. However, stimulated neutrophils produce a burst of active oxygen metabolites, some of which (hypochlorous acid for example) are capable of oxidizing a critical methionine residue in α1-antitrypsin. Oxidized α1-antitrypsin has been shown to have a limited potency as a NE inhibitor and it has been proposed that alteration of this protease/antiprotease balance permits NE to perform its degradative functions in localized and controlled environments.

α1-antitrypsin is a glycoprotein of MW 51,000 with 417 amino acids and 3 oligosaccharide side chains. Human α1-antitrypsin was named anti-trypsin because of its initially discovered ability to inactivate pancreatic trypsin. Human α1-antitrypsin is a single polypeptide chain with no internal disulfide bonds and only a single cysteine residue normally intermolecularly disulfide-linked to either cysteine or glutathione. The reactive site of α1-antitrypsin contains a methionine residue, which is labile to oxidation upon exposure to tobacco smoke or other oxidizing pollutants. Such oxidation reduces the biological activity of α1-antitrypsin; therefore substitution of another amino acid at that position, i.e. alanine, valine, glycine, phenylalanine, arginine or lysine, produces a form of a 1-antitrypsin which is more stable. α1-antitrypsin can be represented by the following formula:

1 MPSSVSWGIL LAGLCCLVPV SLAEDPQGDA AQKTDTSHHD QDHPTFNKIT PNLAEFAFSLYRQLAHQSNS TNIFFSPVSI ATAFAMLSLG TKADTHDEIL EGLNFNLTEI PEAQIHEGFQ ELLRTLNQPD SQLQLTTGNG LFLSEGLKLV DKFLEDVKKL YHSEAFTVNF GDHEEAKKQI NDYVEKGTQG KIVDLVKELD RDTVFALVNY IFFKGKWERP FEVKDTEDED FHVDQVTTVK VPMMKRLGMF NIQHCKKLSS WVLLMKYLGN ATAIFFLPDE GKLQHLENEL THDIITKFLE NEDRRSASLH LPKLSITGTY DLKSVLGQLG ITKVFSNGAD LSGVTEEAPL KLSKAVHKAV LTIDEKGTEA AGAMFLEAIP MSIPPEVKFN KPFVFLMIEQ NTKSPLFMGK VVNPTQK 417

Ciliberto, et al. in Cell 1985, 41, 531-540. The critical amino acid sequence near the carboxyterminal end of α1-antitrypsin is shown in bold and underlined and is pertinent to this invention (details of the sequence can be found for example in U.S. Pat. No. 5,470,970, the full contents of which are incorporated herein by reference).

The normal plasma concentration of ATT ranges from 1.3 to 3.5 mg/ml although it can behave as an acute phase reactant and increases 3-4-fold during host response to inflammation and/or tissue injury such as with pregnancy, acute infection, and tumors. It easily diffuses into tissue spaces and forms a 1:1 complex with a target protease, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen. Humans with circulating levels of α1-antitrypsin less than 15% of normal are susceptible to the development of lung disease, e.g., familial emphysema, at an early age. Familial emphysema is associated with low ratios of α1-antitrypsin to serine proteases, particularly elastase. Therefore, it appears that this inhibitor represents an important part of the defense mechanism against attack by serine proteases.

α1-antitrypsin is one of few naturally occurring mammalian serine protease inhibitors currently approved for the clinical therapy of protease imbalance. Therapeutic α1-antitrypsin has been commercially available since the mid 80s and is prepared by various purification methods (see for example Bollen et al., U.S. Pat. No. 4,629,567; Thompson et al., U.S. Pat. Nos. 4,760,130; 5,616,693; WO 98/56821). Prolastin is a trademark for a purified variant of α1-antitrypsin and is currently sold by Bayer Company (U.S. Pat. No. 5,610,285 Lebing et al., Mar. 11, 1997). Recombinant unmodified and mutant variants of α1-antitrypsin produced by genetic engineering methods are also known (U.S. Pat. No. 4,711,848); methods of use are also known, e.g., (α1-antitrypsin gene therapy/delivery (U.S. Pat. No. 5,399,346 to French Anderson et al.).

The two known cellular mechanisms of action of serine proteases are by direct degradative effects and by activation of G-protein-coupled proteinase-activated receptors (PARs). The PAR is activated by the binding of the protease followed by hydrolysis of specific peptide bonds, with the result that the new N-terminal sequences stimulate the receptor. The consequences of PAR activation depend on the PAR type that is stimulated and on the cell or tissue affected and may include activation of phospholipase C.beta., activation of protein kinase C and inhibition of adenylate kinase (Dery, O. and Bunnett, N. W. Biochem Soc Trans 1999, 27, 246-254; Altieri, D.C. J. Leukoc Biol 1995, 58, 120-127; Dery, O. et al. Am J. Physiol 1998, 274, C1429-C1452).

It is to be understood that the present invention is not limited to the examples described herein, and other serine protease inhibitors known in the art can be used within the limitations of the invention. For example, one skilled in the art can easily adopt inhibitors as described in WO 98/24806, which discloses substituted oxadiazole, thiadiazole and triazole as serine protease inhibitors. U.S. Pat. No. 5,874,585 discloses substituted heterocyclic compounds useful as inhibitors of serine proteases; including: (benzyloxycarbonyl)-L-valyl-N-[1-(3-(5-(3-trifluoromethylbenzyl)-1,2,4-oxa diazolyl)carbonyl)-2-(S)-methylpropyl]-L-prolinamidebenzyloxycarbonyl)-L-valyl-N-[1-(3-(5-(2-phenylethyl)-1,2,4-oxadiazolyl)carbonyl)-2-(S)-methylpropyl]-L-prolinamide; (benzyloxycarbonyl)-L-valyl-N-[1-(3-(5-(2-methoxybenzyl)-1,2,4-oxadiazolyl)carbonyl)-2-(S)-methylpropyl]-L-prolinamide; (benzyloxycarbonyl)-L-valyl-N-[1-(3-(5-(trifluoromethyl)-1,2,4-oxadiazolyl)carbonyl)-2-(S)-methylpropyl]-L-prolinamide; (benzyloxycarbonyl)-L-valyl-N-[1-(3-(5-(methyl)-1,2,4-oxadiazolyl)carbonyl)-2-(S)-Methylpropyl]-L-Prolinamide; (Benzyloxycarbonyl)-L-Valyl-N-[1-(3-(5-(difluoromethyl)-1,2,4-oxadiazolyl)carbonyl)-2-(S)-Methylpropyl]-L-Prolinamide; (Benzyloxycarbonyl)-L-Valyl-N-[1-(3-(5-(benzyl)-1,2,4-oxadiazolyl)carbonyl)-2-(S)-Methylpropyl]-L-Prolinamide; (Benzyloxycarbonyl)-L-Valyl-N-[1-(3-(5-(3-methoxybenzyl)-1,2,4-oxadiazolyl)carbonyl)-2-(S)-Methylpropyl]-L-Prolinamide; (Benzyloxycarbonyl)-L-Valyl-N-[1-(3-(5-(2,6-difluorobenzyl)-1,2,4-oxadiazolyl)carbonyl)-2-(S)-Methylpropyl]-L-Prolinamide; (Benzyloxycarbonyl)-L-Valyl-N-[1-(3-(5-(trans-styryl)-1,2,4-oxadiazolyl)carbonyl)-2-(S)-Methylpropyl]-L-Prolinamide; (Benzyloxycarbonyl)-L-Valyl-N-[1-(3-(5-(trans-4-Trifluoromethylstyryl)-1,2,4-oxadiazolyl)carbonyl)-2-(S)-Methylpropyl]-L-Prolinamide; (Benzyloxycarbonyl)-L-Valyl-N-[1-(3-(5-(trans-4-Methoxystyryl)-1,2,4-oxadiazolyl)carbonyl)-2-(S)-Methylpropyl]-L-Prolinamide; (Benzyloxycarbonyl)-L-Valyl-N-[1-(3-(5-(3-Thienylmethyl)-1,2,4-oxadiazolyl)carbonyl)-2-(S)-Methylpropyl]-L-Prolinamide; (Benzyloxycarbonyl)-L-Valyl-N-[1-(3-(5-(Phenyl)-1,2,4-oxadiazolyl)carbonyl)-2-(S)-methylpropyl]-L-prolinamide; and (Benzyloxycarbonyl)-L-Valyl-N-[1-(3-(5-(3-Phenylpropyl)-1,2,4-oxadiazolyl)carbonyl)-2-(S)-Methylpropyl]-L-Prolinamide. U.S. Pat. No. 5,216,022 teaches other small molecules useful for the practice of this invention, including: Benzyloxycarbonyl-L-valyl-N-[1-(2-[5-(3-methylbenzyl)-1,3,4-oxadiazolyl]carbonyl)-2-(S)-methylpropyl]-L-prolinamide (also known as CE-2072), Benzyloxycarbonyl-L-valyl-N-[1-(2-(3-methylbenzyl)-1,3,4-oxadiazolyl]carbonyl)-2-(S)-methylpropyl]-L-prolinamide; Benzyloxycarbonyl-L-valyl-N-[-(2-(5-(methyl)-1,3,4-oxadiazoly]carbonyl)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; (Benzyloxycarbonyl)-L-valyl-N-[1-(2-(5-(4-Dimethylaminobenzyl)-1,3,4-oxadiazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]carbonyl)-2-(S)-methylpropyl]-L-prolinamide; (Benzyloxycarbonyl)-L-valyl-[[1-(3-(5-(3,4-methylenedioxybenzyl)-1,2,4-oxadiazolyl]carbonyl)-2-(S)-methylpropyl]-L-prolinamide; Benzyloxycarbonyl)-L-valyl-N-[1-(3-(5-(3,5-dimethylbenzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl)-L-valyl-N-[1-(3-(5-(biphenylmethine)-1,2,4-oxadiazolyl]carbonyl)-2-(S)-methylpropyl]-L-prolinamide; (Benzyloxycarbonyl)-L-valyl-N-[1-(3-(5-(4-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4-oxadiazolyl]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene)-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4-oxadiazolyl]carbonyl)-2-(S)-methylpropyl]-L-prolinamide; (Benzyloxycarbonyl)-L-valyl-N-[1-(3-(5-(3,5-diphenylbenzyl]-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide; (Pyrrole-2-carbonyl)-N-(benzyl)glycyl-N-[1-(3-(5-(3-trifluoromethylbenzyl)]-1,2,4-oxadiazolyl)-(S)-methylpropyl]amide; (2S,5S)-5-Amino-1,2,4,5,6,7-hexahydroazepino-[3,2,1]-indole-4-one-carbonyl-N-[1-(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazolyl]carbonyl)-2-(S)-methylpropyl]amide; (R,S)-3-Amino-2-oxo-5-phenyl-1,4,-benzodiazepine-N-[1-(2-(5-(3-methylbenzyl)-1,3,4-oxadiazolyl]carbonyl)-2-(S)-methylpropyl]acetamide; (Benzyloxycarbonyl)-L-valyl-2-L-(2,3-dihydro-1H-indole)-N-[-(2-(5-(3-methylbenzyl)-1,3,4-oxadiazolyl]carbonyl)-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-methylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-.epsilon.-lactam-N-[1-(2-(5-(3-methylbenzyl)-1,3,4-oxadiazolyl]carbonyl)-2-(S)-methylpropyl]acetamide; 3-(S)-(Amino)-.epsilon.-lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholinocarbonyl-butanoyl)amino]-.epsilon.-lactam-N-[1-(2-(5-(3-methylbenzyl)-1,3,4-oxadiazolyl]carbonyl)-2-(R,S)-methylpropyl]acetamide; 6-[4-Fluorophenyl]-.epsilon.-lactam-N-[1-(2-(5-(3-methylbenzyl)-1,3,4-oxadiazolyl]carbonyl)-2-(S)-methylpropyl]acetamide; 2-(2-(R,S)-Phenyl-4-oxothiazolidin-3-yl]-N-[1-(2-(5-(3-methylbenzyl)-1,3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yl oxide]-N-[1-(3-(5-(3-trifluoromethylbenzyl)-1,2,4-oxadiazolyl]carbonyl)-2-(R,S,)-methylpropyl]acetamide; (1-Benzoyl-3,8-quinazolinedione)-N-[1-(2-(5-(3-methylbenzyl)-1,3,4-oxadiazolyl]carbonyl)-2-(S)-methylpropyl]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazol yl]carbonyl)-2-(S)-methylpropyl]acetamide; [(1-Phenyl-3,6-piperazinedione)-N-[1-(3-(5-(3-trifluoromethylbenzyl)-1,2,4-oxadiazolyl]carbonyl)]-2-(S)-methylpropyl]acetamide; 3-[(Benzyloxycarbonyl)amino]-quinolin-2-one-N-[1-(2-(5-(3-methylbenzyl)-1,3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-methylbenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazolyl]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]carbonyl)-2-(S)-methylpropyl]acetamide; 1-Acetyl-3-(4-fluorobenzylidene) piperazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxy benzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,5-dione-N-[1-(2-(5-(3-methylbenzyl)-1,3,4-oxadiazolyl]carbonyl)-2-(S)-methylpropyl]acetamide; 4-[1-Benzyl-3-(R)-benzyl-piperazine-2,5,-dione]-N-[1-(2-[5-(3-methylbenzyl)-1,3,4-oxadiazolyl]carbonyl)-2-methylpropyl]acetamide; 4-[1-Benzyl-3-(S)-benzyl piperazine-2,5,-dione-N-[1-(2-(5-(3-methylbenzyl)-1,3,4-oxadiazolyl]carbonyl)-2-(S)-methylpropyl]acetamide; 4-[1-Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylaminoethyl)-1,2,4-oxadiazolyl]carbonyl)-2-(S)-methylpropyl]acetamide; 4-[1-Methyl-3-(R,S)-phenylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Methyl-3-(R,S)-phenylpiperazine-2,5,-dione]-N-[1-(2-(5-(3-methylbenzyl)-1,3,4-oxadiazolyl]carbonyl)-2-(S)-methylpropyl]acetamide; 4-[1-(4-Morpholino ethyl)3-(R)-benzyl piperazine-2,5,-dione]-N-[1-(2-(5-(3-methylbenzyl)-1,3,4-oxadiazolyl]carbonyl)-2-(S)-methylpropyl]acetamide; 5-(R,S)-Phenyl-2,4-imidazolidinedione-N-[1-(2-(5-(3-methylbenzyl)-1,3,4-oxadiazolyl]carbonyl)-2-(S)-methylpropyl]acetamide; 5-(R)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 1-Benzyl-4-(R)-benzyl-2,5-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,5-imidazolidinedione-N-[1-(3-(5-(3-trifluoromethylbenzyl)-1,2,4-oxadiazolyl]carbonyl)-2-(S)-methylpropyl]acetamide, among others.

Likewise, U.S. Pat. No. 5,869,455 discloses N-substituted derivatives; U.S. Pat. No. 5,861,380 protease inhibitors-keto and di-keto containing ring systems; U.S. Pat. No. 5,807,829 serine protease inhibitor-tripeptoid analogues; U.S. Pat. No. 5,801,148 serine protease inhibitors-proline analogues; U.S. Pat. No. 5,618,792 substituted heterocyclic compounds useful as inhibitors of serine proteases. These patents and PCT publications and others as listed infra are incorporated herein, in their entirety, by reference. Other equally advantageous molecules, which may be used instead of α1-antitrypsin or in combination with α1-antitrypsin are contemplated such as in WO 98/20034 disclosing serine protease inhibitors from fleas. Without limiting to this single reference one skilled in the art can easily and without undue experimentation adopt compounds such as in WO98/23565 which discloses aminoguanidine and alkoxyguanidine compounds useful for inhibiting serine proteases.

Additional Combination Therapies for Treating Viral Diseases Using the Methods of the Invention

In each of the aforementioned aspects and embodiments of the invention, combination therapies other than those enumerated above are also specifically contemplated herein. In particular, the compositions of the present invention may be admininistered with one or more macrolide or non-macrolide antibiotics, anti-bacterial agents, anti-fungicides, anti-viral agents, and anti-parasitic agents, anti-inflammatory or immunomodulatory drugs or agents.

Examples of macrolide antibiotics that may be used in combination with the composition of the present invention include, inter alia, the following synthetic, semi-synthetic or naturally occurring microlidic antibiotic compounds: methymycin, neomethymycin, YC-17, litorin, erythromycin A to F, oleandomycin, roxithromycin, dirithromycin, flurithromycin, clarithromycin, davercin, azithromycin, josamycin, kitasamycin, spiramycin, midecamycin, rokitamycin, miokamycin, lankacidin, and the derivatives of these compounds. Thus, erythromycin and compounds derived from erythromycin belong to the general class of antibiotics known as “macrolides.” Examples of preferred erythromycin and erythromycin-like compounds include: erythromycin, clarithromycin, azithromycin, and troleandomycin.

Additional antibiotics, other than the macrolidic antibiotics described above, which are suitable for use in the methods of the present invention include, for example, any molecule that tends to prevent, inhibit or destroy life and as such, and as used herein, includes anti-bacterial agents, anti-fungicides, anti-viral agents, and anti-parasitic agents. These agents may be isolated from an organism that produces the agent or procured from a commercial source (e.g., pharmaceutical company, such as Eli Lilly, Indianapolis, Ind.; Sigma, St. Louis, Mo.).

For example, the anti-TB antibiotic isoniazid (isonicotinic acid hydrazide) is frequently effective, but isoniazid often causes severe, sometimes fatal, hepatitis. The risk of hepatitis increases with the patient's age. Additionally, isoniazid causes peripheral neuropathy in some recipients in a dose-related fashion. Rifampin, another antibiotic used to treat TB, must be used in conjunction with another drug such as isoniazid. This requirement for combination therapy with rifampin applies to the initial treatment as well as the retreatment of pulmonary TB.

Usually, isoniazid, rifampin, ethambutol and ethionamide are given orally. Streptomycin is typically given intramuscularly. Amikacin is given intramuscularly or intravenously. Clofazimine, which is also used to treat leprosy, is given orally.

Amikacin is a semisynthetic aminoglycoside antibiotic derived from Kanamycin A. For its preparation see U.S. Pat. No. 3,781,268. For a review see Kerridge, Pharmacological and Biochemical Properties of Drug Substances 1:125-153, M. E. Goldberg, ed. (1977). Amikacin is usually administered intramuscularly or intravenously. For additional information including clinical pharmacology, indications, side effects and dosages, see the Physicians Desk Reference, 42 ed. (1988) at pages 744-746 (hereinafter, PDR).

Clofazimine is an antibacterial agent also known as LAMPRENE.RTM. For its preparation, see Barry, et at., Nature 179:1013 (1957). For a review see Karat, et al., Brit. Med. J. 3:175 (1971). Clofazimine is generally given orally. For additional information including clinical pharmacology, precautions and dosages, see the PDR at page 982.

Ethionamide is an antibacterial agent also known as AMIDAZINE.RTM. and TRECATOR.RTM. See British Patent No. 800,250. This drug is typically given orally. For further information including precautions and dosages, see the PDR at page 2310.

Ciprofloxacin is a broad spectrum synthetic antibacterial agent for oral usage. It is also known as CIPRO.RTM. It is typically given in total daily dosages of 500 to 1,000 milligrams which is usually given in 2 equal doses in 24 hours. For further information see the PDR (1989) at pages 1441-1443. other member of this fluoroquinolone class of antibiotics include ofloxacin, levofloxacin, troveofloxacin, pefloxacin, gatifloxacin, and moxifloxacin.

Other examples of anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, oxazalidinones, and fluoroquinolones. Examples of antibiotic agents include, but are not limited to, Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefmetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazfdime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

Anti-fungal agents include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents include, but are not limited to, valgancyclovir, amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole (sulfamethoxazole/trimethoprim), and pentamidine isethionate.

In another aspect, in the method of the present invention, one may, for example, supplement the composition by administration of a therapeutically effective amount of one or more an anti-inflammatory or immunomodulatory drugs or agents. By “immunomodulatory drugs or agents”, it is meant, e.g., agents which act on the immune system, directly or indirectly, e.g., by stimulating or suppressing a cellular activity of a cell in the immune system, e.g., T-cells, B-cells, macrophages, or antigen presenting cells (APC), or by acting upon components outside the immune system which, in turn, stimulate, suppress, or modulate the immune system, e.g., hormones, receptor agonists or antagonists, and neurotransmitters; immunomodulators can be, e.g., immunosuppressants or immunostimulants. By “anti-inflammatory drugs”, it is meant, e.g., agents which treat inflammatory responses, i.e., a tissue reaction to injury, e.g., agents which treat the immune, vascular, or lymphatic systems.

Anti-inflammatory or immunomodulatory drugs or agents suitable for use in this invention include, but are not limited to, interferon derivatives, e.g., betaseron, .beta.-interferon; prostane derivatives, e.g., compounds disclosed in PCT/DE93/0013, e.g., iloprost, cicaprost; glucocorticoid, e.g., cortisol, prednisolone, methylprednisolone, dexamethasone; immunsuppressives, e.g., cyclosporine A, FK-506, methoxsalene, thalidomide, sulfasalazine, azathioprine, methotrexate; lipoxygenase inhibitors, e.g., zileutone, MK-886, WY-50295, SC-45662, SC-41661A, BI-L-357; leukotriene antagonists, e.g., compounds disclosed in DE 40091171 German patent application P 42 42 390.2; WO 9201675; SC-41930; SC-50605; SC-51146; LY 255283 (D. K. Herron et al., FASEB J. 2: Abstr. 4729, 1988); LY 223982 (D. M. Gapinski et al. J. Med. Chem. 33: 2798-2813, 1990); U-75302 and analogs, e.g., described by J. Morris et al., Tetrahedron Lett. 29: 143-146, 1988, C. E. Burgos et al., Tetrahedron Lett. 30: 5081-5084, 1989; B. M. Taylor et al., Prostaglandins 42: 211-224, 1991; compounds disclosed in U.S. Pat. No. 5,019,573; ONO-LB-457 and analogs, e.g., described by K. Kishikawa et al., Adv. Prostagl. Thombox. Leukotriene Res. 21: 407-410, 1990; M. Konno et al., Adv. Prostagl. Thrombox. Leukotriene Res. 21: 411-414, 1990; WF-11605 and analogs, e.g., disclosed in U.S. Pat. No. 4,963,583; compounds disclosed in WO 9118601, WO 9118879; WO 9118880, WO 9118883, antiinflammatory substances, e.g., NPC 16570, NPC 17923 described by L. Noronha-Blab. et al., Gastroenterology 102 (Suppl.): A 672, 1992; NPC 15669 and analogs described by R. M. Burch et al., Proc. Nat. Acad. Sci. USA 88: 355-359, 1991; S. Pou et al., Biochem. Pharmacol. 45: 2123-2127, 1993; peptide derivatives, e.g., ACTH and analogs; soluble TNF-receptors; TNF-antibodies; soluble receptors of interleukines, other cytokines, T-cell-proteins; antibodies against receptors of interleukins, other cytokines, and T-cell-proteins.

In yet another aspect of the present invention, for each of the above-recited methods, the one or more substances exhibiting Tubercin and/or SSM activity or a functional derivative thereof may themselves be administered as an adjuvant, wherein the therapeutically effective amount of the one or more substances exhibiting Tubercin and/or SSM activity or a functional derivative thereof acts as an immunostimulant or immunomodulator to be used either alone or in conjunction with one or more of the pharmaceutical drugs listed infra.

In yet another aspect of the present invention, for each of the above-recited methods of the present invention, the therapeutically effective amount of the one or more substances exhibiting Tubercin and/or SSM activity or a functional derivative thereof may themselves be administered as an adjuvant in vaccine preparations to improve vaccine responses to all known bacterial, viral or parasitic antigen preparations, wherein the therapeutically effective amount of the one or more substances exhibiting Tubercin and/or SSM activity or a functional derivative thereof acts as an immunostimulant or immunomodulator to be used either alone or in conjunction with one or more of the pharmaceutical drugs. Representative examples of pharmaceutical drugs that may be used in accordance with the of the present invention include the following, as well as each specific indication specifically listed for use with each drug as if set forth herein in their entirety and as described in the Physician's Desk Reference (PDR)(the entire contents of which are incorporated herein by reference) listed in the Physician's Desk References.

The therapeutic agents of the instant invention may be used for the treatment of animal subjects or patients, and more preferably, mammals, including humans, as well as mammals such as non-human primates, dogs, cats, horses, cows, pigs, guinea pigs, and rodents.

Fusion Proteins

In each of the aforementioned aspects and embodiments of the invention, fusion polypeptides are also specifically contemplated herein.

In one embodiment, fusion polypeptides of the invention are produced by recombinant DNA techniques. Alternative to recombinant expression, a fusion polypeptide of the invention can be synthesized chemically using standard peptide synthesis techniques. The present invention also provides compositions that comprise a fusion polypeptide of the invention and a pharmaceutically acceptable carrier, excipient or diluent.

In each of the above-recited methods, the substance exhibiting Tubercin and/or SSM activity or a functional derivative thereof may be part of a fusion polypeptide, wherein said fusion polypeptide or conjugate fusion polypeptide comprises a substance exhibiting Tubercin and/or SSM activity or a functional derivative thereof and an amino acid sequence heterologous to said substance exhibiting Tubercin and/or SSM activity or a functional derivative thereof.

Among the particular fusion polypeptides or conjugate fusion polypeptides of the invention are, for example, fusion polyeptides or conjugate fusion polypeptides that comprise the substance exhibiting Tubercin and/or SSM activity or a functional derivative thereof.

The fusion polypeptides or conjugate fusion polypeptides of the invention can be such that the heterologous amino acid sequence comprises a human immunoglobulin constant region, such as a human IgG1 constant region, including a modified human IgG1 constant region wherein the IgG1 constant region does not bind Fc receptor and/or does not initiate antibody-dependent cellular cytotoxicity (ADCC) reactions.

In particular, in one embodiment the fusion protein or conjugate fusion polypeptide comprises a heterologous sequence that is a sequence derived from a member of the immunoglobulin protein family, for example, comprise an immunoglobulin constant region, e.g., a human immunoglobulin constant region such as a human IgG1 constant region. The fusion protein or conjugate fusion polypeptide can, for example, comprise a portion of a substance exhibiting Tubercin and/or SSM activity or a functional derivative thereof fused or conjugated with the amino-terminus or the carboxyl-terminus of an immunoglobulin constant region, as disclosed, e.g., in U.S. Pat. No. 5,714,147, U.S. Pat. No. 5,116,964, U.S. Pat. No. 5,514,582, and U.S. Pat. No. 5,455,165. In those embodiments in which all or part of a substance exhibiting Tubercin and/or SSM activity or a functional derivative thereof of the invention is fused with sequences derived from a member of the immunoglobulin protein family, the FcR region of the immunoglobulin may be either wild-type or mutated. In certain embodiments, it is desirable to utilize an immunoglobulin fusion protein that does not inteact with a Fc receptor and does not initiate ADCC reactions. In such instances, the immunoglobulin heterologous sequence of the fusion protein can be mutated to inhibit such reactions. See, e.g., U.S. Pat. No. 5,985,279 and WO 98/06248.

The heterologous amino acid sequence of the fusion polypeptides or conjugate fusion polypeptides utilized as part of the present invention can also comprise an amino acid sequence useful for identifying, tracking or purifying the fusion polypeptide, e.g., can comprise a FLAG or a His tag sequence. The fusion polypeptide or conjugate fusion polypeptide can further comprise an amino acid sequence containing a proteolytic cleavage site which can, for example, be useful for removing the heterologous amino acid sequence from the substance exhibiting Tubercin and/or SSM activity or a functional derivative thereof or from the a substance exhibiting Tubercin and/or SSM activity or a functional derivative thereof/α1-antitrypsin or inhibitor of serine protease derivative or synthetic mimic sequence conjugate fusion polypeptide.

In particular, the heterologous amino acid sequence of the fusion polypeptides or conjugate fusion polypeptides of the present invention can also comprise an amino acid sequence useful for identifying, tracking or purifying the fusion polypeptide, e.g., can comprise a FLAG (see, e.g., Hoop, T. P. et al., Bio/Technology 6, 1204-1210 (1988); Prickett, K. S. et al., BioTechniques 7, 580-589 (1989)) or a His tag (Van Reeth, T. et al., BioTechniques 25, 898-904 (1998)) sequence. The fusion polypeptide or conjugate fusion polypeptides can further comprise an amino acid sequence containing a proteolytic cleavage site which can, for example, be useful for removing the heterologous amino acid sequence from the substance exhibiting. Tubercin and/or SSM activity or a functional derivative thereof.

In yet another embodiment, the fusion polypeptide or conjugate fusion polypeptide comprises a GST fusion protein in which the substance exhibiting Tubercin and/or SSM activity or a functional derivative thereof or the substance exhibiting Tubercin and/or SSM activity or a functional derivative thereof/α1-antitrypsin or inhibitor of serine protease derivative or synthetic mimic sequence conjugate fusion polypeptide of the invention is fused to the C-terminus of GST sequences. Such a fusion protein can facilitate the purification of a recombinant polypeptide of the invention. In those embodiments in which a GST, FLAG or His Tag fusion constructs is employed in the construction of the substance exhibiting Tubercin and/or SSM activity or a functional derivative thereof fusion proteins, proteolytic cleavage sites may be optionally introduced at the junction of the fusion moiety and the substance exhibiting Tubercin and/or SSM activity or a functional derivative thereof to enable separation of the substance exhibiting Tubercin and/or SSM activity or a functional derivative thereof from the fusion moiety subsequent to purification of the substance exhibiting Tubercin and/or SSM activity or a functional derivative thereof. Such enzymes, and their cognate recognition sequences, include, for example, without limitation, Factor Xa, thrombin and enterokinase. Typical fusion expression vectors include pGEX (Pharmacia Biotech Inc.; Smith and Johnson (1988) Gene 67:31-40), pMAL (New England Biolabs, Beverly, Mass.) and pRIT5 (Pharmacia, Piscataway, N.J.) which may be used to fuse glutathione S-transferase (GST), maltose E binding protein, or protein A, respectively, to the target substance exhibiting Tubercin and/or SSM activity or a functional derivative thereof.

Expression vectors can routinely be designed for expression of a fusion polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn 10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the alpha-fetoprotein promoter (Campes and Tilghman (1989) Genes Dev. 3:537-546).

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

Modes of Administration

Modes of administration of the various therapeutic agents used in the invention are exemplified below. However, the agents can be delivered by any of a variety of routes including: by injection (e.g., subcutaneous, intramuscular, intravenous, intraarterial, intraperitoneal), by continuous intravenous infusion, cutaenously, dermally, transdermally, orally (e.g., tablet, pill, liquid medicine), by implanted osmotic pumps (e.g., Alza Corp.), by suppository or aerosol spray.

The peptide conjugate-based serine protease inhibitors used in the Tubercin and/or SSM-protein/peptide (for example, AAT) combination therapy of the present invention are used as therapeutic agents in the treatment of a physiological (especially pathological) condition caused in whole or part, by excessive serine protease activity. The peptide conjugates may be administered as free peptides or pharmaceutically acceptable salts thereof. Those skilled in the art of biochemical synthesis will recognize that for commercial-scale quantities of peptide conjugates, such peptide conjugates are preferably prepared using recombinant DNA techniques, synthetic techniques, or chemical derivatization of biologically or chemically synthesized peptides.

The peptide conjugate-based serine protease inhibitors used in the Tubercin and/or SSM-protein/peptide (for example, AAT) combination therapy may be prepared by any suitable synthesis method such as originally described by Merrifield, J. Am. Chem. Soc., 85, p 2149 (1963). Synthetic peptides which exhibit inhibitory activity toward serine proteases and methods for preparing and using same are disclosed for example in U.S. Pat. Nos. 4,829,052, 5,157,019 to Glover; U.S. Pat. No. 5,420,110 to Miller; U.S. Pat. No. 4,963,654 Katunuma as incorporated herein by reference. In addition, methods for the addition of saccharides to protein are known to those skilled in the art. For example, addition of sialyl Lewis acid X to antibodies for targeting purposes is described in U.S. Pat. No. 5,723,583; and modification of oligosaccharides to form vaccines is described in U.S. Pat. No. 5,370,872. A general strategy for forming protein-saccharide conjugates is outlined in U.S. Pat. No. 5,554,730. Moreover, additional synthesis methods may be found in United States Patent Application 20040214228, the entire contents of which are incorporated herein by reference in their entirety.

The terms used herein conform to those found in Budavari, Susan (Editor), “The Merck Index” An Encyclopedia of Chemicals, Drugs, and Biologicals; Merck & Co., Inc. The term “pharmaceutically acceptable salt” refers to those acid addition salts or metal complexes of the Tubercin and/or SSM compounds and or functional equivalent thereof or peptide conjugate-based serine protease inhibitors used in the Tubercin and/or SSM-AAT combination therapy which do not significantly or adversely affect the therapeutic properties (e.g. efficacy, toxicity, etc.) of the compounds or peptides conjugatest. The compounds or peptide conjugates should be administered to individuals as a pharmaceutical composition, which, in most cases, will comprise the compounds or peptides and/or pharmaceutical salts thereof with a pharmaceutically acceptable carrier. The term “pharmaceutically acceptable carrier” refers to those solid and liquid carriers, which do not significantly or adversely affect the therapeutic properties of the compounds or peptide conjugates.

The pharmaceutical compositions containing compounds or peptide conjugates of the present invention may be administered to individuals, particularly humans, either intravenously, subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of inhibitors of serine proteases. Transdermal administration is accomplished by application of a cream, rinse, gel, etc. capable of allowing the inhibitors of serine proteases to penetrate the skin and enter the blood stream. Parenteral routes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy, tracheostomy, endotracheal tube, or metered dose or continuous inhaler. In addition, osmotic pumps may be used for administration. The necessary dosage will vary with the particular condition being treated, method of administration and rate of clearance of the molecule from the body.

Although the Tubercin and/or SSM compounds described herein and/or their functional derivatives may be administered as the pure chemicals, it is preferable to present the active ingredient as a pharmaceutical composition. The invention thus further provides the use of a pharmaceutical composition comprising one or more Tubercin and/or SSM compounds and/or their functional derivatives and/or a pharmaceutically acceptable salt thereof, together with one or more pharmaceutically acceptable carriers therefore and, optionally, other therapeutic and/or prophylactic ingredients. The carrier(s) must be acceptable in the sense of being compatible with the other ingredients of the composition and not deleterious to the recipient thereof.

Pharmaceutical compositions include those suitable for oral or parenteral (including intramuscular, subcutaneous, cutaneous, inhaled and intravenous) administration. The compositions may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical compositions suitable for oral administration may be presented as discrete unit dosage forms such as hard or soft gelatin capsules, cachets or tablets, each containing a predetermined amount of the active ingredient; as a powder or as granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art., e.g., with enteric coatings.

Oral liquid preparations may be in the form of, for example, aqueous or oily suspension, solutions, emulsions, syrups or elixirs, or may be presented as a dry product for constitution with water or another suitable vehicle before use. Such liquid preparations may contain conventional additives such as suspending agents, emulsifying agents, non-aqueous vehicles (which may include edible oils), or preservative. The compounds may also be formulated for parenteral administration (e.g., by injection, for example, bolus injection or continuous infusion) and may be presented in unit dose form in ampoules, pre-filled syringes, small bolus infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds or peptide conjugates may be formulated as ointments, creams or lotions, or as the active ingredient of a transdermal patch. Suitable transdermal delivery systems are disclosed, for example, in Fisher et al. (U.S. Pat. No. 4,788,603) or Bawas et al. (U.S. Pat. Nos. 4,931,279, 4,668,504 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. No. 4,140,122, 4,383,529, or 4,051,842. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention may also contain other adjuvants such as flavorings, coloring, antimicrobial agents, or preservatives.

It will be further appreciated that the amount of the Tubercin and/or SSM and/or a functional derivative compound, or an active salt or derivative thereof, required for use in treatment will vary not only with the particular salt selected but also with the route of administration, the nature of the condition being treated and the age and condition of the patient and will be selected, ultimately, at the discretion of the attending physician.

A pharmaceutical composition of the invention contains an appropriate pharmaceutically acceptable carrier as defined supra. These compositions can take the form of solutions, suspensions, tablets, pills, capsules, powders, sustained-release formulations and the like. Suitable pharmaceutical carriers are described in Remington's Pharmaceutical Sciences 1990, pp. 1519-1675, Gennaro, A. R., ed., Mack Publishing Company, Easton, Pa. The Tubercin and/or SSM compounds described herein and/or their functional derivatives either alon or in combination with the serine protease inhibitor molecules of the invention can be administered in liposomes or polymers (see, Langer, R. Nature 1998, 392, 5). Such compositions will contain an effective therapeutic amount of the active compound together with a suitable amount of carrier so as to provide the form for proper administration to the subject.

In general, the compounds of the present invention are conveniently administered in unit dosage form; for example, containing 5 to 2000 mg, conveniently 10 to 1000 mg, most conveniently, 50 to 500 mg of active ingredient per unit dosage form.

Desirable blood levels may be maintained by continuous infusion to provide about 0.01-5.0 mg/kg/hr or by intermittent infusions containing about 0.4-20 mg/kg of the active ingredient(s). Buffers, preservatives, antioxidants and the like can be incorporated as required.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular pharmaceutical compound or analogue thereof of the present invention, the route of administration, the severity of the condition being treated, and the condition and prior medical history of the patient being treated. However, it is within the skill of the art to start doses of the pharmaceutical compound at levels lower than required to achieve the desired therapeutic effect and to gradually increase the dosage until the desired effect is achieved.

The pharmaceutical compositions of the present invention can be used in both veterinary medicine and human therapy. The magnitude of a prophylactic or therapeutic dose of the pharmaceutical composition of the invention in the acute or chronic management of pain associated with above-mentioned diseases or indications will vary with the severity of the condition to be treated and the route of administration. The dose, and perhaps the dose frequency, will also vary according to the age, body weight, and response of the individual patient. In general, the total daily dose range of the pharmaceutical composition of this invention is generally between about 1 to about 100 mg, preferably about 1 to about 20 mg, and more preferably about 1 to about 10 mg of active compound per kilogram of body weight per day are administered to a mammalian patient. If desired, the effective daily dose may be divided into multiple doses for purposes of administration, e.g. two to four separate doses per day.

Alternatively, the total daily dose range of the active ingredient of this invention ishould be sufficient to increase the serum concentraiton of the proease inhibtor by 10-100 micromolar.

It is intended herein that by recitation of such specified ranges, the ranges cited also include all those dose range amounts between the recited range. For example, in the range about 1 and 100, it is intended to encompass 2 to 99, 3-98, etc, without actually reciting each specific range. The actual preferred amounts of the active ingredient will vary with each case, according to the species of mammal, the nature and severity of the particular affliction being treated, and the method of administration.

It is also understood that doses within those ranges, but not explicitly stated, such as 30 mg, 50 mg, 75 mg, etc. are encompassed by the stated ranges, as are amounts slightly outside the stated range limits.

The actual preferred amounts of the active ingredient will vary with each case, according to the species of mammal, the nature and severity of the particular affliction being treated, and the method of administration.

In general, the pharmaceutical compositions of the present invention are periodically administered to an individual patient as necessary to improve symptoms of the particular disease being treated. The length of time during which the compositions are administered and the total dosage will necessarily vary with each case, according to the nature and severity of the particular affliction being treated and the physical condition of the subject or patient receiving such treatment.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know, with no more than routine experimentation, how and when to interrupt, adjust, or terminate therapy in conjunction with individual patient response.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

### EXAMPLES

The following specific examples are provided to better assist the reader in the various aspects of practicing the present invention. As these specific examples are merely illustrative, nothing in the following descriptions should be construed as limiting the invention in any way. Such limitations are, or course, defined solely by the accompanying claims.

### Example One

### Introduction/Materials and Methods

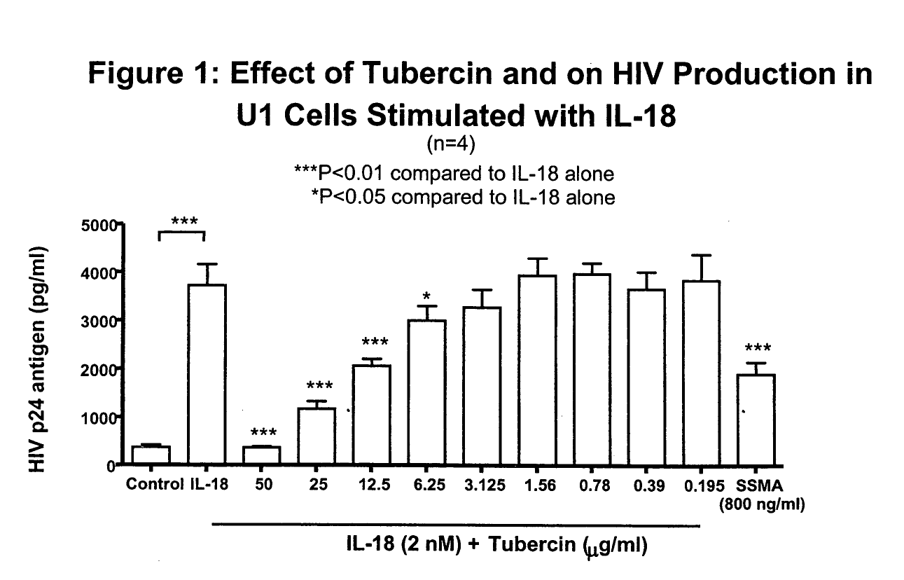

HIV is a human retrovirus that integrates into the genome of the host. After integrating into the host genome as a provirus, HIV can be induced to replicate from this latent reservoir following stimulation with several endogenous and exogenous pro-inflammatory molecules. Examples of endogenous pro-inflammatory molecules include certain cytokines like interleukin (IL)-1, IL-18, and tumor necrosis factor (TNF). Exogenous pro-inflammatory substances include the bacterial cell wall product lipopolysaccharide (LPS or endotoxin) and the gram-positive cell-wall substance lipoteichoic acid. U1 cells are a human cell line derived from human monocytic U937 cells that contain 2 copies of Human Immunodeficiency Virus Type 1 (HIV) incorporated into the cell nucleus as a provirus. Upon stimulation with any of several pro-inflammatory mediators, the amount of expressed virus can be dramatically increased. Therefore, these cells constitute an in vitro model of chronic HIV infection. As shown in FIG. 1, these cells were cultured for 24 hrs at a density of 1×10⁶ cells per ml in 24-well polystyrene tissue culture plates (Falcon). Cells were cultured in medium consisting of RPMI tissue culture medium (Cellgro, Herndon, Va.) with 10% vol/vol fetal calf serum (FCS, Life Technologies, Grand Island, N.Y.) with penicillin/streptomycin (100 units/ml/100 μg/ml, Life Technologies). After the 24 hrs of incubation (37° C., 5% CO₂ atmosphere), the cell cultures were lysed using Triton-X-100 (Sigma, St Louis, Mo.) and the total amount of HIV p24 quantified using a p24 ELISA (Beckman-Coulter).

Results:

As shown in FIG. 1, U1 cells were cultured in medium alone (Control), with IL-18 as a stimulus alone (MBL, 2 nM final concentration), or with IL-18 in the presence of Tubercin or SSMA (supplied by Mr. Colm King) (SSMA is SSM or specific substance of Maruyama) at the final concentrations shown on the horizontal axis. Tubercin or SSMA was added to the cultures 2 hrs prior to the addition of the IL-18 stimulus. As shown, Tubercin inhibited IL-18-induced HIV dose dependently. A maximum inhibitory effect of 100% compared to IL-18 alone was observed at a concentration of 50 μg/ml Tubercin. Significant inhibition due to Tubercin was observed down to a minimum concentration of 6.25 μg/ml. In addition, SSMA at 800 ng/ml also inhibited HIV substantially in these cultures.

Discussion:

These results establish an anti-HIV activity for Tubercin and SSMA in a chronically HIV-infected human cell line. The inhibition observed was potent (up to 100%) and the inhibitory effect demonstrated a dose-response. Statistically significant inhibition of IL-18-induced HIV was observed for Tubercin concentrations as low as 6.25 μg/ml.

### Example Two

### Introduction/Materials and Methods

In the same U1 cell line used in FIG. 1, the generalizability of the Tubercin and SSMA inhibitory activity was assessed. To accomplish this, U1 cells were stimulated to produce HIV using lipopolysaccharide (LPS, also referred to as endotoxin) instead of IL-18. Using the same protocol as described for FIG. 1, U1 cells were stimulated in the absence or the presence of Tubercin or SSMA to assess the effect of Tubercin on LPS-induced HIV.

Results:

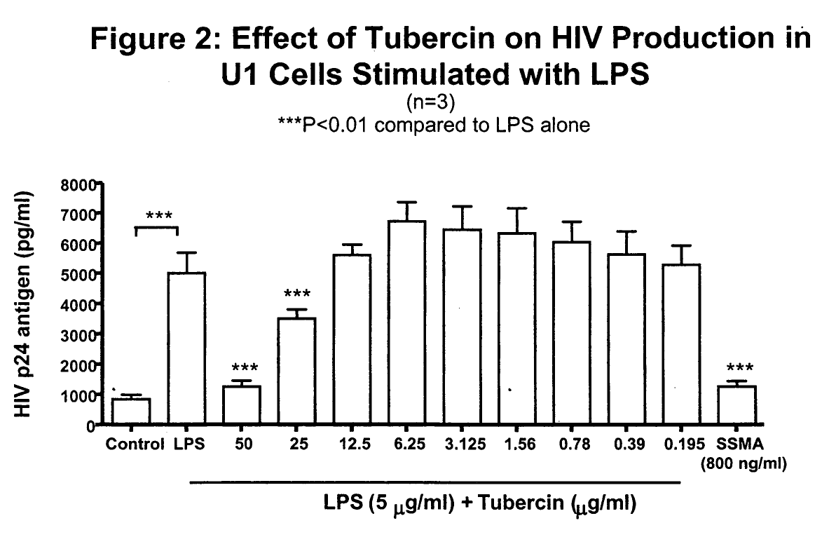

As shown in FIG. 2, the presence of Tubercin at final concentrations 50 or 25 μg/ml significantly inhibited the amount of HIV stimulated by 5 μg/ml LPS. Additionally, the presence of SSMA at 800 ng/ml substantially inhibited HIV production in these experiments.

Discussion:

These results extend the data shown in FIG. 1 to demonstrate that Tubercin and SSMA can inhibit stimulated HIV. It is thus suggested that the Tubercin and SSMA inhibitory effect is not stimulus-specific.

### Example Three

### Introduction/Materials and Methods

The effect of Tubercin and SSMA on U1 cell viability and on U1 cell replication was examined. The results observed in FIGS. 1 and 2 potentially represent a true suppressive anti-HIV effect. On the other hand, these data could have resulted from a toxic effect of the Tubercin, or to an antiproliferative Tubercin effect. To exclude these possibilities, cell counting in a hemacytometer and used the trypan blue vital stain was used to determine possible toxic or anti-proliferative Tubercin effects. Proliferation and toxicity studies were conducted in triplicate U1 cell cultures conducted for 24 hrs in the absence (Control) or the presence of Tubercin or SSMA. After 24 hrs of incubation, the cells were counted in a blinded fashion using a hemacytometer, and the vital dye trypan blue was used to assess cell viability (cells that were infiltrated with the trypan blue stain are not viable, and blue-staining cells were counted as dead).

Results:

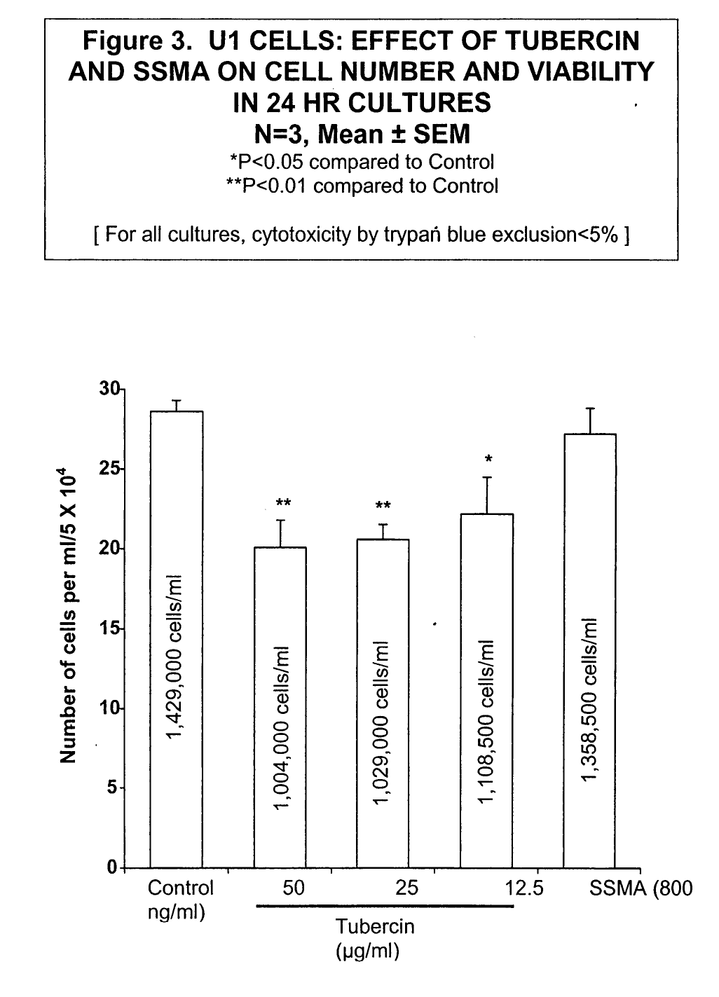

As depicted in FIG. 3, Tubercin dose-dependently reduced the number of U1 cells present (compared to Control cultures conducted in the absence of Tubercin) after 24 hr of incubation. Thus, Tubercin demonstrated a statistically significant anti-proliferative effect in U1 cells. The maximum suppression of proliferation was approximately 30% at 50 μg/ml Tubercin. However, there was no observed cytotoxicity by trypan blue exclusion at any Tubercin concentration tested. In the cultures exposed to SSMA, there was no notable effect on U1 cell proliferation, and there was no SSMA effect on U1 cell viability as assessed by trypan blue exclusion.

Discussion:

These data indicate that Tubercin possessed a modest but statistically significant anti-proliferative effect in U1 cells. However, there was no associated U1 cell cytotoxicity due to Tubercin. The maximum magnitude of anti-proliferative activity (approximately 30% at 50 μg/ml Tubercin) cannot account for the antiretroviral effect of Tubercin observed in the U1 cell cultures, as the maximum HIV suppression obtained was nearly 100% in U1 cells stimulated with IL-18 or LPS (see FIGS. 1 and 2). These results establish the absence of toxicity of Tubercin in U1 cells. Therefore, the Tubercin-induced suppression of HIV in stimulated U1 cells (FIGS. 1 and 2) was due to a true antiretroviral Tubercin effect.

In addition, there is an antiproliferative effect of Tubercin that was significant and dose-dependent. While this antiproliferative activity of Tubercin may account for a part of the Tubercin anti-HIV activity, it is insufficient to explain the full (100%) anti-HIV effects that were observed in FIGS. 1 and 2. It is of special note that Tubercin is reported to possess antineoplastic (anti-cancer) activity in vitro and in vivo. Although this effect is thought to arise from up-regulation of the host anti-neoplastic immune response, the data in FIG. 3 suggest that a complementary anti-neoplastic effect may derive from a direct anti-proliferative effect on neoplastic cells.

### Example Four

### Introduction/Materials and Methods

The anti-HIV results obtained in U1 cells in FIGS. 1 and 2 show that Tubercin inhibits stimulated HIV production in the chronically HIV-infected U 1 cell line. There are two possible limitations of these experiments: a) U1 cells are a human immortalized cell line and thus differ from HIV-infected natural cells that exist in infected persons, b) U1 cells contain HIV as a provirus and it is therefore a pure production model of HIV synthesis; there is no de novo HIV infection in these cells.

To assess the Tubercin effect in primary (natural) human cells, human peripheral blood mononuclear cells (PBMC) were used that were isolated from the blood of healthy human volunteers. Heparinized blood was obtained from healthy volunteers and the PBMC isolated using centrifugation through ficoll-hypaque. The PBMC were then infected using an M-tropic strain of HIV, using 100 TCID₅₀ HIV per million PBMC using a protocol previously described in the inventors laboratory (Shapiro L, Pott G B, Ralston A H. Alpha-1-antitrypsin inhibits human immunodeficiency virus type 1. FASEB J, 15:115-122, 2001). The HIV-infected PBMC were cultured in R3 medium consisting of RPMI medium, 10% (vol/vol) FCS, 5% (vol/vol) IL-2, and penicillin/streptomycin (100 units/ml/100 μg/ml). After infection, PBMC were washed in RPMI medium to remove free virus. An aliquot of cells was lysed (1% vol/vol triton-X-100) and the total HIV was quantified; this was designated the T=0 sample that represented the amount of virus in the cultures prior to incubation. HIV-infected PBMC were aliquoted into wells of 24-well polystyrene tissue culture plates (Falcon) at a cell density of 1×10⁶ cells per ml in a final volume of 0.5 ml. After 3 days of incubation (37° C., 5% CO₂), the cell cultures were lysed with 1% (vol/vol) triton-X-100 and the cultures stored at −70° C. until assayed for HIV using a p24 ELISA.

Results:

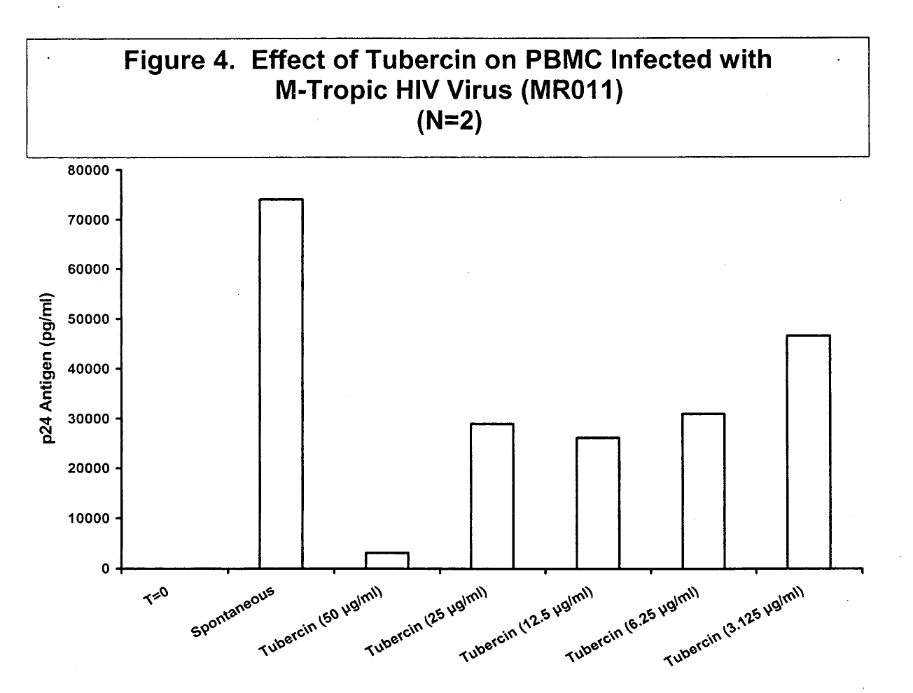

FIG. 4 depicts results using PBMC from two separate donors. As shown, the 3 day cell cultures synthesized a substantial amount of new virus compared to the amount of virus initially added to the cultures (shown in the graph as Spontaneous HIV production and T=0 levels, respectively). As shown in FIG. 4, the addition of Tubercin to the cultures induced a substantial and dose-dependent inhibition of HIV production in infected PBMC derived from the two individuals. In fact, the magnitude of inhibition was nearly 100% at 50 μg/ml Tubercin.

Discussion:

These results extend the scope of anti-HIV activity of Tubercin to primary infected cells. Since this model of HIV production more closely reflects condition in vivo, it increases the plausibility that Tubercin inhibits HIV production in infected patients. Furthermore, since HIV production in infected PBMC represents serial rounds of both infection and production, these results suggest a possible inhibitory role for Tubercin in HIV infection.

### Example Five

### Introduction/Materials and Methods

The toxicity of Tubercin in HIV-infected PBMC, as well as the specificity of the Tubercin antiretroviral effect in these primary infected cells were assessed. These two issues were assessed simultaneously by quantifying the amount of cytokines produced in one of the HIV-infected PBMC cultures shown in FIG. 4.

Results:

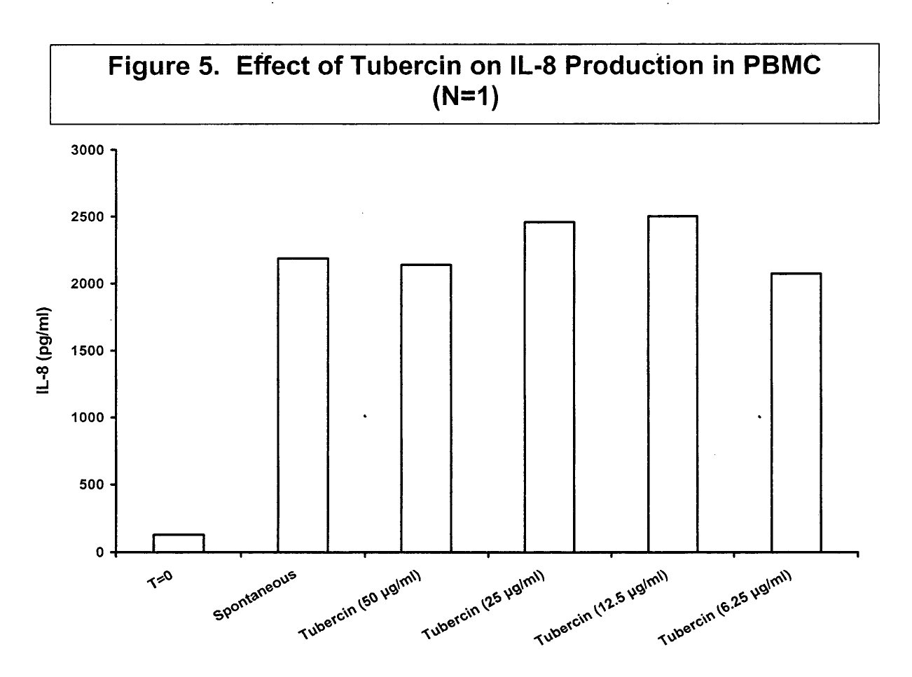

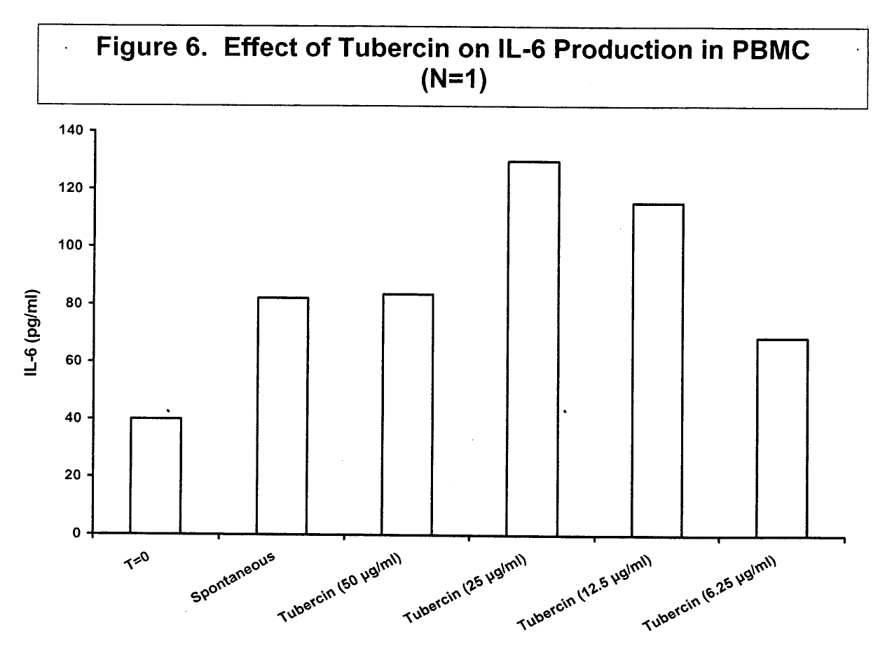

FIG. 5 shows IL-8 measured in this culture, and FIG. 6 shows IL-6 measured in this same culture. As graphed, after 3 days of culture, both IL-8 (FIG. 5) and IL-6 (FIG. 6) increased in the cultures conducted in medium alone (Spontaneous culture) compared to T=0 (amount present in the cultures prior to the 3 days of incubation). As depicted for both IL-8 and IL-6, the presence of Tubercin for the 3 days of culture did not reduce the amount of either cytokine, compared to Spontaneous cultures.

Discussion:

These results lead to two conclusions about Tubercin:

a. Tubercin is not toxic to HIV-infected PBMC. If Tubercin were toxic to these cells, then there would have been reduced amounts of cytokines after 3 days of culture, since damaged or dead cells cannot synthesize cytokines.

b. The anti-HIV activity is specific, in that Tubercin suppressed HIV production substantially (FIG. 4), but had no concomitant suppressive effect on IL-8 or on IL-6. In fact, Tubercin demonstrated a small inducing effect on IL-6 production at concentrations 25 and 12.5 μg/ml (FIG. 6).

### Example 6

### MAGI-CCR-5 Cell Infection

Introduction/Materials and Methods:

The experiments conducted in U1 monocytic cells and in HIV-infected PBMC assess HIV production (U 1 cells) or HIV infection and production (PBMC). In order to assess the effect of Tubercin on HIV infection, MAGI (multinuclear activation of a galactosidase indicator)-CCR-5 cells were used. These cells are human HeLa cells that express all of the cell surface HIV receptors required to permit entry of virus into the cell interior. Once HIV is inside the cell, the virus integrates into the nucleus. As soon as the viral proteins are expressed in the cells, the HIV tat protein interacts with a genomic reporter construct that induces the synthesis of □-galactosidase. A developing solution is then used to stain the □-galactosidase-containing cells. This assay constitutes a system that indicates early events in the HIV infectious life cycle.

MAGI-CCR-5 cells (NIH AIDS Research and Reference Reagent Program, NIAID) were aliquoted into 24-well polystyrene plates (Falcon) at 4×10⁴ per well in a 1.0 ml volume. After 24 h of incubation (37° C., 5% CO₂), all medium (RPMI, 10% v/v fetal calf serum, penicillin/streptomycin) was removed from each well and 200 μl fresh medium was added without or with Tubercin at the final concentrations shown on the horizontal axis. Three hundred TCID₅₀ of AO18A strain of HIV-1 and 20 μg/ml DEAE dextran in 200 μl medium were then added to the cell-containing wells. To evaluate background reporter activation, a separate cell-containing well received DEAE dextran in medium without virus. After 2 h of incubation, medium was added to each culture to adjust the final volume to 1,000 μl, and the cultures were incubated for 48 h. Medium was then aspirated, the cells were fixed, and a 1-galactosidase staining solution was added. After 50 min of incubation, a blinded count of pigmented (reporter-activated) cells under a microscope was conducted.

Results:

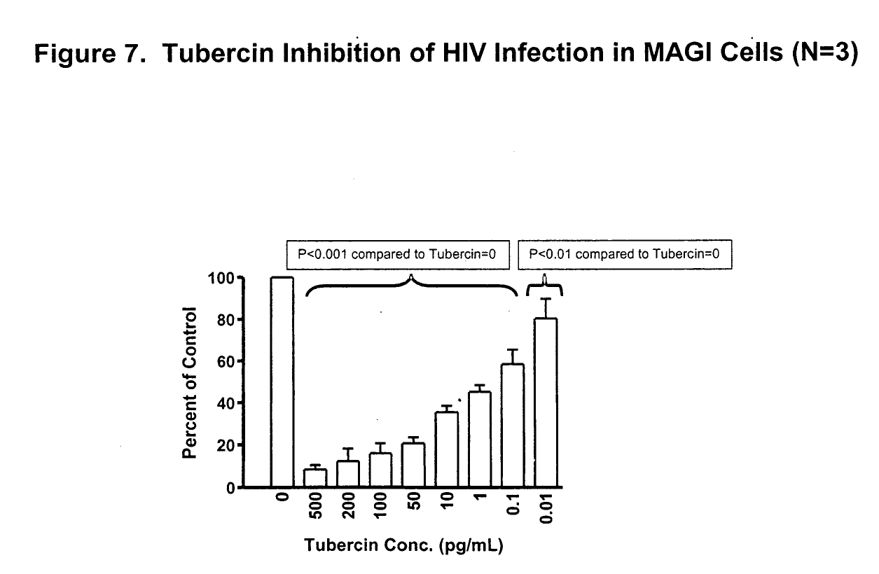

As shown in FIG. 7 below, cells cultured in medium alone produced a large HIV infection, indicated by 100%. In MAGI-CCR-5 cell cultures exposed to Tubercin at the concentrations shown on the horizontal axis resulted in a dose-dependent and statistically significant reduction in infection of the cells with virus. Significant inhibition was observed using all Tubercin concentrations tested (P<0.01 or P<0.001). It is remarkable that even Tubercin concentrations as low as 10 femtograms/ml significant blocked HIV infection. The maximum degree of inhibition was observed using 500 pg/ml Tubercin, which resulted in approximately 90% blockade of HIV infectivity.

Discussion:

These data demonstrated that Tubercin can inhibit the earliest stages of HIV infection in an in vitro model of HIV infection. In conjunction with the U1 cell data described previously, these results establish that Tubercin is capable of blocking both HIV infection and HIV production in individual cells. These results have implications for possible clinical use for Tubercin as an anti-retroviral agent in infected humans. It is suggested that Tubercin may possess anti-HIV effects at extremely low concentrations in infected patients. Concentrations in the range 1-500 pg/ml may suffice to inhibit HIV production in vivo.

### Example 7

### Generalizability Statement

It is likely that the Tubercin anti-HIV effect extends to inhibition of additional viruses. This conjecture is reasonable given that Tubercin appears to affect the cell itself and does not target virus-specific substances. Most compelling are the data describing an anti-HIV Tubercin effect in U1 cells (see above). In this model purely of production of HIV from a provirus state, the Tubercin effect likely involves alteration in the signal transduction events that eventuate in virus production. Specifically, it is likely that Tubercin interferes with signal transductiion pathways that are associated with IL-18 and LPS stimulation of U 1 cells that eventuate in virus production (see FIGS. 7 and 8 above). It is important to note that many (if not all) viruses require cell-associated signal transduction activity to enable viral infection and synthesis. Therefore, the inhibitory effect of Tubercin on HIV infection and production likely extends to any, if not all other viruses that infect human cells.

### Example 8

In this example, for each of the methods of the present invention, the therapeutically effective amount of the one or more substances exhibiting Tubercin and/or SSM activity and/or Z-100 activity or a functional derivative thereof and a pharmaceutically acceptable excipient thereof is administered to a subject in need thereof in conjunction with a therapeutically effective amount of one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof taken either alone or in any combination thereof. In each method, the virus, viral indication, bacterial diseases or variant thereof is effectively inhibited in said mammal.

### EQUIVALENTS

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for treating a viral infection in a mammal comprising administering to a subject in need thereof of a therapeutically effective amount of a composition comprising a substance exhibiting Tubercin and/or SSM activity or a functional derivative thereof in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof; and a pharmaceutically acceptable excipient.

2. A method of relieving or ameliorating the pain or symptoms associated with one or more viral diseases or indications in a mammal suffering from one or more viral diseases or indications comprises administering to the mammal in need thereof a therapeutically effective pain or symptom-reducing amount of a pharmaceutical composition comprising effective amounts of a substance exhibiting Tubercin and/or SSM activity or a functional derivative thereof in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof, and a pharmaceutically acceptable carrier or excipient, wherein said Tubercin and/or SSM activity or a functional derivative thereof substance is sufficient to relieve or ameliorate the pain or symptoms associated with one or more viral diseases or indications.

3. A method of relieving or ameliorating the pain or symptoms associated with one or more viral diseases or indications in a mammal suffering from one or more viral diseases or indications comprises administering to the mammal in need thereof a therapeutically effective pain or symptom-reducing amount of a pharmaceutical composition comprising effective amounts of a substance exhibiting Tubercin and/or SSM activity or a functional derivative thereof in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgernanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof; and a pharmaceutically acceptable carrier or excipient, wherein said Tubercin and/or SSM activity or a functional derivative thereof substance is sufficient to relieve or ameliorate the pain or symptoms associated with one or more mycobacterial diseases or indications.

4. The method of claim 1, wherein the therapeutically effective amount of one or more substances exhibiting Tubercin and/or SSM activity or functional derivatives thereof may be administered to a subject in need thereof in conjunction with a therapeutically effective amount of one or more anti-inflammatory compounds and/or a therapeutically effective amount of one or more immunomodulatory agents, and in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof.

5. The method according to claim 4, wherein the anti-inflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

6. The method according to claim 1, wherein the therapeutically effective amount of one or more substances exhibiting Tubercin and/or SSM activity or a functional derivative thereof may be administered to a subject in need thereof in conjunction with one or more antimicrobial or antiviral compositions or any combination thereof.

7. The method according to claim 1, wherein the reduction or inhibition of pain and/or symptoms associated with the one or more of viral indications is on the order of about 10-20%, 30-40% 50-60%, or 75-100% reduction or inhibition.

8. A method for preventing a symptom of a given viral infection in a subject thought to be at risk for exposure to a given viral infection comprising admininstering to the subject a pharmaceutically effective amount of a substance exhibiting Tubercin and/or SSM activity or a functional derivative thereof in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof, wherein said substance exhibiting Tubercin and/or SSM activity or a functional derivative thereof substance inhibits the attachment of a given virus to one or more viral receptors, and wherein if the subject is exposed to the virus, a symptom of said exposure is prevented.

9. A method for preventing a symptom of a given viral infection in a subject suspected of having been exposed to a given viral infection comprising admininstering to the subject a pharmaceutically effective amount of a substance exhibiting Tubercin and/or SSM activity or a functional derivative thereof in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof, wherein said substance exhibiting Tubercin and/or SSM activity or a functional derivative thereof substance inhibits the attachment of a given virus to one or more viral receptors, and wherein if the subject is exposed to the virus, a symptom of said exposure is prevented.

10. A method for ameliorating a symptom of a given viral infection in a subject in need of said amelioration comprising admininstering to the subject a pharmaceutically effective amount of a substance exhibiting Tubercin and/or SSM activity or a functional derivative thereof in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethyl germanium sesquioxide, a synthesized organo germanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide, derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof, wherein said substance exhibiting Tubercin and/or SSM activity or a functional derivative thereof substance inhibits the attachment of a given virus to one or more viral receptors.

11. The method according to claim 7, wherein the symptom of viral infection that is inhibited or prevented is selected from the group consisting of malaise, fever, chills, rhinitis, diarrhea, atopic eczema, encephalitis, keratoconjunctivitis, pharyngitis, gingivostomatitis, herpetic hepatitis, recurrent orofacial mucocutaneous lesions or herpes labialis, small pox skin sores, chicken pox skin sores, erythema multifomme, idiopathic burning mouth, aphthous ulceration, Behcet's syndrome, mononucleosis, Burkitt's lymphoma, primary effusion lymphomas, multiple myeloma, angioimmunoblastic lymphadenopathy, Castleman's disease, acquired immune deficiency syndrome (AIDS)-related lymphoma, post-transplantation lymphoproliferative disease, Hodgkin's disease, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, malignant mesothelioma, angiosarcoma, Kaposi's sarcoma, angiolymphoid hyperplasia, prostatic neoplasm, cervical cancer, neoplasms of the vulva, retinoblastoma, Li-Fraumeni syndrome, Gardner's syndrome, Werner's syndrome, nervoid basal cell carcinoma syndrome, neurofibromatosis type 1, polyneuropathies, motor neuropathies, sensory neuronopathies, polyradiculoneuropathies, autonomic neuropathies, focal or multifocal cranial neuropathies, radiculopathies, plexopathies typically resulting from tumor infiltration, sexually or perinatally transmitted herpes disease, malaise, fever, dry cough, myalgias, and chest pains, ventilatory compromise, sweating, widening of the mediastimum on radiographic studies, edema of the neck and chest, necrotizing mediastinal lymphadenitis, non-pitting edema, eschar, nausea, vomiting, fever, abdominal pain, bloody diarrhea, mucosal ulcerations, hemorrhagic mesenteric lymphadenitis or any combination thereof

12. The method according to claim 1, wherein the pharmaceutical compositions are administered orally, systemically, via an implant, intravenously, topically, intrathecally, by inhalation, or nasally.

13. The method according to claim 1, wherein the substance exhibiting Tubercin and/or SSM activity or a functional derivative thereof may be part of a fusion polypeptide, wherein said fusion polypeptide comprises a substance exhibiting Tubercin and/or SSM activity or a functional derivative thereof and an amino acid sequence heterologous to said substance exhibiting Tubercin and/or SSM activity or a functional derivative thereof substance.

14. A method for treating or prophylaxing one or more viral diseases by inhibiting proinflammatory cytokine production in a patient in need thereof by administering a substance exhibiting Tubercin and/or SSM activity or a functional derivative thereof to said patient in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof.

15. The method of claim 14, wherein the cytokine inhibited is IL-1, TNFalpha, IL-18, or nitric oxide, or any combination thereof.

16. A method for inhibiting a bacterial opportunistic infection in a mammal comprising admininstering to the subject a pharmaceutically effective amount of a substance exhibiting Tubercin and/or SSM activity or a functional derivative thereof either alone or in combination with one or more immune enhancers, wherein the immune enhancers comprise one or more of Glycyrrhizin (GR), consisting of one molecule of glycyrrhetic acid and two molecules of glucuronic acid, is an active component of Glycyrrhiza glabra roots, Ge-132, Carboxyethylgermanium sesquioxide, a synthesized organogermanium compound, SMANCS (polystyrene-co-maleic acid conjucated neocarzinostatin), Krestin (PSK), hot water extract of mycelium of Criolus versicolor, Picibanil (OK-432), a whole cell preparation of streptococcus pyogenes, Lentinan, a B-1,3-glucan extracted from fruit body of Lentinus ebodes, KS-2 (peptidomannan), Muramyl dipeptide (active component of LPS), ATSO, an antitumor polysaccharide from Coriolus versicolor, Levamisole (synthesized compound), 9-Methylstreptimidone (antibiotics), Schizophyllan, a polysaccharide derived from culture medium of Schizophyllum commune, Corynebacterium parvum, Mushroom extract, aqueous extract of Lentinus edodes, a kind of Japanese edible mushroom, Dextran phosphate, TH69, a lyophilized whole cell preparation of Streptococcus faecalis, Shigyaku-to (TJS-109), traditional Chinese herbal medicine, Kanzo-bushi-to (TJS-038) one of the Japanese Kampo medicines originating from traditional Chinese herbal medicine, Keishi-Ka-Kei-To, a traditional Chinese herbal medicine, and analogues thereof, wherein the bacterial opportunistic infection comprises Candidiasis (Thrush), Chronic mucocutaneous candidiasis, Candida vulvovaginitis, Dry mouth (xerostomia), Diabetes mellitus, Cytomegalovirus (CMV), Herpes simplex viruses, Oral herpes, Genital herpes, Herpes Zoster, Malaria, Mycobacterium avium complex (MAC or MAI), Pneumocystis pneumonia (PCP), Toxoplasmosis (Toxo), Tuberculosis (TB), Pneumocystis jirovecii, Pneumocystis carinii, Candida albicans infection, Staphylococcus aureus, Streptococcus pyogenes, Streptococcus pneumoniae, Pseudomonas aeruginosa, Acinetobacter baumanni, Toxoplasma gondii, Cytomegalovirus, Cytomegalovirus retinitis, Aspergillus sp., Aspergillosis, Penicillium marneffei, Miliary TB, MRSA, Invasive candidiasis, Histoplasmosis, Disseminated strongyloidiasis, Legionellosis, Respiratory syscytial virus, Hepatitis, Hepatitis A, Hepatitis B, Salmonellosis, Syphilis and Neuroshyphilis, Bacillary angiomatosis, Coccidioidomycosis, Cryptococcal meningitis, Kaposi's Sarcoma, Systemic Non-Hodgkin's Lymphoma, Primary CNS lymphoma, Cryptosporidiosis, Isosporiasis, Microsporidiosis, Human Papiloma Virus (HPV, genital warts, cervical cancer), Molluscum Contagiosum, Oral Hairy Leukoplakia (OHL), Progressive Multifocal Leukoencephalopathy (PML), AIDS Dementia Complex (ADC), Peripheral Neuropathy, Apthous Ulcers, Malabsorption, Systemic inflammatory response syndrome (SIRS), E. faecalis, Methicillin-resistant Staphylococcus aureus (MRSA), CLP-induced sepsis, Salmonella typhimurium, Trypanosoma cruzi, Lymphocytic choriomeningitis virus, Listeria monocytogenes, Herpetic encephalomyelitis, Cryptococcal encephalitis, Human cytomegalovirus, Herpes simplex virus type 2, Influenza virus, Coronavirus, HIV, Poliovirus, Newcastle disease virus, Vesicular stomatitis virus, Ehrlich carcinoma, Sarcoma-180, or any variants thereof.
